(19) United States
(12) Patent Application Publication
(10) Pub. No.: US 2018/0363056 A1
(43) Pub. Date: Dec. 20, 2018

(54) METHODS FOR DIRECT MEASUREMENT OF MICRORNA INHIBITION AND MIMICRY

(71) Applicant: Regulus Therapeutics Inc., San Diego, CA (US)

(72) Inventors: John R. Androsavich, San Diego, CA (US); B. Nelson Chau, San Diego, CA (US); Ryan R. Galasso, San Diego, CA (US)

(73) Assignee: Regulus Therapeutics Inc., San Diego, CA (US)

(21) Appl. No.: 15/754,061

(22) PCT Filed: Sep. 2, 2016
(86) PCT No.: PCT/US2016/050136

§ 371 Date: Feb. 21, 2018

Related U.S. Application Data
(60) Provisional application No. 62/214,447, filed on Sep. 4, 2015.

(51) Int. Cl. C12Q 1/6883

(52) U.S. Cl. CPC C12Q 1/6883; C12Q 2600/178; C12Q 2600/136

(57) ABSTRACT

The disclosure includes methods of determining the activity of an inhibitor of a target microRNA (miRNA) or of a mimic of a target microRNA. Determination of polysome occupancy in treated and control samples followed by comparing the occupancies can be used to determine a displacement value for the inhibitor or mimic of the target miRNA. The displacement value reflects the extent of a change in the levels of the target miRNA (which may include the mimic if applicable) in polysomal and non-polysomal compartments of a sample or a shift of the target miRNA between polysomal and non-polysomal compartments of a sample and can indicate the activity of the inhibitor toward the target miRNA or of the mimic toward a target RNA.

### CROSS-REFERENCE TO RELATED APPLICATIONS

This application claims the benefit of priority of U.S. Provisional Patent Application No. 62/214,447, filed Sep. 4, 2015, which is incorporated by reference herein in its entirety for all purposes.

### FIELD OF INVENTION

Provided herein are methods for the direct measurement of microRNA inhibition and of microRNA mimicry.

### BACKGROUND

Aberrant microRNA (microRNA) activity has been implicated in a number of diseases such as inflammatory disease, fibrosis, and cancer. Inhibition of overactive microRNA has been shown to improve disease outcome in a number of preclinical animal studies (See, for example, Gomez et al., JCI, 2015; 125: 141-156; Chau et al., Sci Trans Med., 2012; 4: 121ra118; Trajkovski et al., Nature, 2011; 474: 649-653).

In the case of chemically modified anti-miR oligonucleotides designed to sterically inhibit microRNA via complementary base pairing, a common approach for drug-target engagement has been to assess the amount of detectable microRNA remaining after anti-miR treatment. High-affinity anti-miRs generally do not induce microRNA degradation but rather sequester cognate microRNA in a stable duplex (See, for example, Davis et al., Nucleic Acids Res, 2009; 37: 70-77; Hogan et al. PloS one, 2014; 9: e100951; and Torres et al., RNA, 2011; 17: 933-943). This duplex is, in theory, resistant to hybridization by reverse transcription primers, therefore measuring microRNA levels with and without anti-miR treatment is expected to provide an estimate of percent inhibition. Although this approach, referred to herein as RT-interference, has frequently been reported in literature (See, e.g., Denzler et al., Mol Cell, 2014; 54:766-776), its accuracy has not been demonstrated and some have questioned its validity (See, e.g. Stenvang et al., Silence, 2012; 3:1; and Davis et al., Nucleic Acids Res, 2009; 37: 70-77).

A more functional yet distal measurement of anti-miR drug activity is assessing derepression of downstream microRNA regulated genes. Currently, identifying and validating microRNA targets as PD biomarkers is non-trivial. Although developments in computational predication (Grimson et al., Mol Cell, 2007; 27: 91-105; Garcia et al., Nat Struct Mol Biol., 2011; 18:1139-1146; Krek et al., Nat Genet., 2005; 37:495-500; Kertesz et al., Nat Genet., 2007; 39: 1278-1284) and biochemical methods (Androsavich et al., Nucleic Acids Res., 2014; 42: 6945-6955; Beitzinger et al., Methods Mol Biol., 2014; 732: 153-167; Karginov et al., PNAS, 2007; 104: 19291-19296) are welcome advancements, the validation process continues to present challenges in vivo. Additionally, the small magnitude of target gene derepression observed (1.1-2 fold) (Ebert et al., Cell, 2012; 149: 515-524), especially when combined with biological and technical variability, is often too narrow of a window for making confident drug discovery decisions such as lead compound selection and early go/no-go determination. Since target repression (and reciprocal derepression) is generally positively correlated with microRNA levels relative to those of target mRNAs (Denzler et al., Mol Cell, 2014; 54:766-776; Garcia et al., Nat Struct Mol Biol., 2011), this problem is exacerbated in studies using healthy animals where basal levels of microRNA targets-of-interest are, often by definition, low compared to disease models. Additional mechanisms may also dampen microRNA activity in the absence of stress (Leung and Sharp, Mol Cell, 2010; 40: 205-215; Mendell and Olson, Cell, 2012; 148:1172-1187).

### SUMMARY OF INVENTION

Provided herein are methods for determining the activity of an inhibitor of a target microRNA comprising:
a. determining a polysome occupancy in a treated sample;
b. determining a polysome occupancy in a control sample;
c. comparing the polysome occupancy in the treated sample to the polysome occupancy in the control sample to determine a displacement value for the inhibitor of the target microRNA, thereby determining the activity of the inhibitor of the target microRNA.

In certain embodiments, the inhibitor of the target microRNA is a modified oligonucleotide. In certain embodiments, the inhibitor of the target microRNA is a small molecule.

In certain embodiments, in sample treated with an inhibitor of a target microRNA, at least one downstream target of the microRNA is not measurably derepressed in the treated sample. In certain embodiments, at least two, at least three, at least four, or at least five downstream targets of the microRNA are not measurably derepressed in the treated sample. In certain embodiments, at least one downstream target of the microRNA has previously been determined to not be measurably derepressed following treatment with the same inhibitor of the target microRNA. In certain embodiments, at least two, at least three, at least four, or at least five downstream targets of the microRNA have previously been determined to not be measurably derepressed following treatment with the same inhibitor of the target microRNA.

Provided herein are methods for determining the activity of a mimic of a target microRNA comprising:
a. determining a polysome occupancy in a treated sample;
b. determining a polysome occupancy in a control sample;
c. comparing the polysome occupancy in the treated sample to the polysome occupancy in the control sample to determine a displacement value for the mimic of the target microRNA, thereby determining the activity of the mimic of the target microRNA.

In certain embodiments, the mimic is a double-stranded compound. In certain embodiments, the mimic is a single-stranded compound.

In certain embodiments, in a sample treated with a mimic of a target microRNA, at least one downstream target of the mimic of a target microRNA is not measurably repressed in the treated sample. In certain embodiments, at least two, at least three, at least four, or at least five downstream targets of the mimic of the target microRNA are not measurably repressed in the treated sample. In certain embodiments, at least one downstream target of the mimic of the target microRNA has previously been determined to not be measurably derepressed following treatment with the same mimic. In certain embodiments, at least two, at least three, at least four, or at least five downstream targets of the mimic of the target microRNA have previously been determined to not be measurably derepressed following treatment with the same mimic of the target microRNA.

In certain embodiments, the treated sample and control sample are each derived from a collection of cells. In certain embodiments, the treated sample and control sample are each derived from a tissue.

In certain embodiments, a lysate is prepared from the treated sample, and from the control sample. In certain embodiments, the lysate from the treated sample is separated into one or more polysomal compartments and one or more non-polysomal compartments, and the lysate from the control sample is separated into one or more polysomal compartments and one or more non-polysomal compartments.

In certain embodiments, separating comprises:
a. layering the lysate on a sucrose gradient;
b. centrifuging the sucrose gradient; and
c. collecting one or more fractions of the sucrose gradient.

In certain embodiments, at least one fraction of the sucrose gradient is a polysomal compartment. In certain embodiments, the methods comprise identifying one or more polysomal compartments of the separated lysate.

In certain embodiments, the method comprises determining at least one of the polysome occupancies by (i) separating at least one polysomal compartment of a sample from at least one non-polysomal compartment of that sample, (ii) quantifying the target microRNA in the separated polysomal compartment, and (iii) quantifying a reference RNA in the same separated polysomal compartment.

In certain embodiments, quantifying the target microRNA and/or quantifying the reference RNA comprises quantitative PCR, spectrophotometry, electrophoresis, hybridization, precipitation, fluorometry, colorimetry, densitometry, scintillation counting, autoradiography, or a combination of two or more of the foregoing procedures.

In certain embodiments, quantifying the target microRNA and/or quantifying the reference RNA comprises (a)(i) contacting the target microRNA and/or the reference RNA with a detectably labeled oligonucleotide to form a detection complex, and (ii) detecting the detection complex; or (b)(i) contacting the microRNA and/or the reference RNA with a primer, (ii) performing a nucleic acid synthesis reaction in which the primer is extended, and (iii) detecting nucleic acid produced by the nucleic acid synthesis reaction.

In certain embodiments, the displacement value is determined by subtracting the logarithm of the control sample poly some occupancy from the logarithm of the treated sample polysome occupancy.

In certain embodiments, the displacement value is represented by the following formula in which polysome occupancies are expressed as logarithmic values:

D=[polysome occupancy,treated sample]−[polysome occupancy,control sample]

In certain embodiments, the displacement value is determined as a logarithm of the quotient of control sample polysome occupancy divided by treated sample polysome occupancy.

In certain embodiments, the displacement value is represented by the following formula in which polysome occupancies are expressed as absolute values:

$$D = {log}_{2} ( \frac{polysome occupancy , control sample}{polysome occupancy , treated sample} )$$

In certain embodiments, determining the polysome occupancy of the target microRNA in the treated sample comprises
i. measuring the amount of the target microRNA in the treated sample by quantitative PCR to generate a target microRNA Ct value for the treated sample;
ii. measuring the amount of a reference RNA in the treated sample by quantitative PCR to generate a reference RNA Ct value for the treated sample; and
iii. subtracting the reference RNA Ct value for the treated sample from the target microRNA Ct value for the treated sample, wherein the resulting value is the polysome occupancy for the treated sample; and
determining the polysome occupancy of the target microRNA in the control sample comprises
iv. measuring the amount of the target microRNA in the control sample by quantitative PCR to generate a target microRNA Ct value for the control sample;
v. measuring the amount of a reference microRNA in the control sample by quantitative PCR to generate a reference RNA Ct value for the control sample; and
vi. subtracting the reference RNA Ct value for the control sample from the target microRNA Ct value for the control sample, wherein the resulting value is the polysome occupancy for the control sample.

In certain embodiments, determining the displacement value for the inhibitor of the target microRNA comprises subtracting the polysome occupancy of the control sample from the polysome occupancy of the treated sample, wherein the resulting value is the displacement value for the target microRNA.

In certain embodiments, determining the displacement value for the mimic of the target microRNA comprises subtracting the polysome occupancy of the control sample from the polysome occupancy of the treated sample, wherein the resulting value is the displacement value for the mimic of the target microRNA.

In certain embodiments, the polysome occupancy is the amount of a target microRNA associated with a polysomal compartment normalized to the amount of a reference RNA associated with the same polysomal compartment.

In certain embodiments, the polysome occupancy of a sample treated with a mimic is the polysome occupancy of the target microRNA and the mimic of the target microRNA.

In certain embodiments, the reference RNA is selected from a reference mRNA and a reference microRNA. In certain embodiments, the reference RNA is let-7.

In certain embodiments, the downstream target of the microRNA is a messenger RNA.

In certain embodiments, the treated sample and/or the control sample is an accessible tissue. In certain embodiments, the treated sample and the control sample are each adipose tissue.

In certain embodiments, the target microRNA is miR-103 or miR-107 or both miR-103 and miR-107. In certain embodiments, the target microRNA is miR-21. In certain embodiments, the target microRNA is miR-17. In certain embodiments, the target microRNA is a member of the let-7 family. In certain embodiments, the target microRNA is let-7a. In certain embodiments, the target microRNA is a member of the miR-34 family. In certain embodiments, the target microRNA is miR-34a.

### BRIEF DESCRIPTION OF DRAWINGS

FIG. 1. Schematic overview of available methods for measuring pharmacodynamics of anti-miR drugs. Following in vivo dosing tissue is harvested and processed for total RNA using phenol/chloroform (Trizol) and cartridge purification. RNA can be analyzed with RT-qPCR using gene specific primers to measure functional changes in microRNA regulated gene expression, or using microRNA primers to measure direct PD/drug-target engagement (TE) by RT-interference. An alternative strategy for measuring direct PD reported herein is the microRNA Polysome Shift Assay, which adds a fractionation step before RNA processing and microRNA RT-qPCR.

FIG. 2. RT-interference poorly reflects ratios of anti-miR inhibited microRNA. (A) An in vitro annealing experiment was used to assess the ability of RT-interference to distinguish between free and anti-miR bound microRNA. Synthetic microRNA guide strand was annealed with cognate anti-miR in increasing ratios. Polyacrylamide gel electrophoresis (PAGE) was used to confirm duplex formation as ground truth for benchmarking RT-interference. (B) Annealing efficiency of miR-122:anti-miR-122 as assessed by PAGE. A Cy3 version of miR-122 guide strand was used for detection. Duplex formation at each A:M ratio was determined by relative densitometry of lower (single stranded) and upper (double stranded) bands. Dashed line shows fit by least-squares linear regression (R²=0.972). (C) RT-interference results using miR-122 (1e7 copies/ng RNA) and anti-miR-122 combination. Grey dashed line represents expected loss of miR-122 for each A:M ratio shown. NTC=no template control. Data are normalized to samples without anti-miR (A:M=0). Error bars represent s.d. for n=3 replicates. (D) PAGE annealing assessment of miR-21 and anti-miR-21 combination (R²=0.970). (E-F) RT-interference results with (E) low miR-21 copy number (1e6 copies/ng RNA) and (F) high miR-21 (1e7 copies/ng RNA).

FIG. 3. Measurement of miR-122 inhibition by microRNA polysome shift assay (miPSA). (A) Representative UV absorbance trace of liver lysates fractionated by ultracentrifugation through sucrose gradients. Fractions were collected from top (light)-to-bottom (dense) and are marked by their leading edge. For 15 fraction gradients, fractions 7-15 were identified as containing polysomes. In all plots, grey bar along x-axis marks polysome fractions. (B-D) Anti-miR-122 causes a specific dose-dependent shift of miR-122 out of polysome fractions. (B) RNA was isolated from each fraction and RT-qPCR was used to quantify microRNA levels. Shown are the proportions of miR-122 (open shapes) and let-7d (filled shapes) in each fraction 24 hours after treatment with anti-miR-122 or saline. For each microRNA, data were normalized to total microRNA detected across all fractions and are expressed as percent per fraction. (C) Cumulative percent miR-122 (black bars; on left for each sample) or let-7d (grey bars; on right for each sample) in polysome fractions. (D) The same data shown in (B) now showing fold-change displacement of miR-122 per fraction for each dose level of anti-miR-122 or saline. Positive displacement values are interpreted as displacement or loss of microRNA and negative values are interpreted as enrichment or gain of microRNA, relative to let-7d reference in log₂ scale. (D) Final summary plot of average loss of miR-122 from polysome fractions for each treatment at 24 hours. ** p<0.01, *** p<0.001, ns=non-significant by one-way ANOVA with Tukey's post-hoc test. (E) (Upper graph) Quantification by HPLC-FL of anti-miR-122 (black bars) or anti-miR-21 (grey bars) in the top 5 fractions of an 8 fraction gradient. For both anti-miRs, the bulk of the oligo remained at the top of the gradient, consistent with its low molecular weight. For anti-miR-122, no anti-miR was detectable in polysome fractions above the limit of detection (LOD, marked by dotted line for each anti-miR). For anti-miR-21, trace amounts were detected in polysome fractions close to the LOD. ULOQ=upper limit of quantification. Error bars represent s.d. for n=3 biological replicates. †One of these samples was below the LOD. (Lower table; top row) Distribution of anti-miR-21 detected in the gradient represented as percent per fraction measured. (Lower table; bottom row) Anti-miR-21 distribution in each measured fraction estimated as a percent of total anti-miR-21 in tissue based on FIG. 9.

FIG. 4. Comparison of miPSA with other pharmacodynamics methods. (A) Comparison of miPSA displacement (black triangle, dashed line) and mRNA expression changes of miR-122 target genes Aldoa (black circle, solid line) and Cd320 (grey square, solid line) at 24 hours and 7 days post-injection. (B) Time course of plasma concentrations of anti-miR-122 measured by hybridization ELISA following injection at 0.3 mpk (light grey triangle), 1.0 mpk (grey square), and 3.0 mpk (black circle). LLOQ=lower limit of quantification. (C) Correlation of miPSA vs Aldoa (left; Pearson r=0.947, p<0.0001) or Cd320 (right; Pearson r=0.952, p<0.0001). Line represents linear regression. (D) Relationship between negative RT-interference and target gene derepression in log₂ scale. Data shown fit with non-linear hyperbolic equation (R²=0.882 for Aldoa; R²=0.9074 for Cd320), which fit better than linear regression (R²=0.642 for Aldoa; R²=0.684 for Cd320). For all plots, error bars represent s.e.m. for n≥3 replicates.

FIG. 5. Measurement of miR-21 inhibition by miPSA in non-stressed tissue. (A) Comparison of miR-21 miPSA displacement (black squares) and target gene derepression (empty circles) in liver as a function of dose 7 days post-injection. Target gene derepression represents a composite score of summed log₂ fold-changes for three miR-21 seed-matched genes: Spg20, Rnf167, and Taf7. Error bars represent s.e.m. for n=4 animals per group. (B) Correlation between miPSA and composite target gene score in liver across two independent experiments with a total n=90 animals. Inset shows correlations for the individual miR-21 target genes. For each plot, Pearson correlation coefficients are shown with linear regression fits (dotted black lines). (C) Comparison of miR-21 displacement in liver (black squares) and kidney (empty circles) from the same animals. Error bars represent s.e.m. for n=4-5 animals per group. (D) Time course of miR-21 displacement in kidney in dose response at day 1 (empty squares, light grey dashed line), day 4 (dark grey filled squares, solid line), day 7 (empty circles, black solid line), and day 10 (black filled circles, solid line). Error bars represent s.e.m. for n=7 animals per group. (E) RT-interference measured with RNA input isolated from intact liver pieces (black bars; on left for each sample) or S16 liver lysates (grey bars; on right for each sample). The same data is represented on both graphs, with y-axes shown in log (left) and linear (right) scales. Data represent linear fold-changes in miR-122 normalized to let-7d and PBS samples. Error bars represent s.d. for n=3 per group. (F) Correlation between target gene composite score and negative RT-interference measured from intact liver (left; black) and S16 lysates (right; grey).

FIG. 6. Assessment of anti-miR cross-reactivity using miPSA. (A) Alignment of mature microRNA sequences showing a common seed between miR-17 and family members miR-20b and miR-106a. Although not part of the miR-17 family, miR-18a has a near identical seed sequence apart from a single A to G change at position 4. This base could theoretically form a G:U wobble with anti-miR-17. (B) Heat map showing displacement of miR-17 family members and other microRNAs in response to anti-miR-17 transfected into cultured cells in dose response at 1, 3, 10, 30, and 100 nM compared to mock (PBS). Darker shades of blue represent greater mean microRNA displacement as depicted in the key.

FIG. 7. Heat map showing response of miR-21 target gene signature to anti-miR-21 (green), mismatch control (yellow), or saline (black). Expression values (Z-score) by RNAseq are shown with each row scaled mean=0. Rows are labeled by Gene Name-Entrez ID.

FIG. 8. (A) Representative UV absorbance trace of kidney lysates fractionated by ultracentrifugation through sucrose gradients. Fractions were collected from top (light)-to-bottom (dense) and are marked by their leading edge. Fractions 3-7 were identified as containing polysomes. (B-C) Anti-miR-21 causes a specific dose-dependent shift of miR-21 out of kidney polysome fractions. (B) RNA was isolated from each fraction and RT-qPCR was used to quantify miRNA levels. Shown are the proportions of miR-21 (empty shapes) and let-7d (filled shapes) in each fraction 7 days after treatment with anti-miR-21 or saline. For each miRNA, data were normalized to total miRNA detected across all fractions and are expressed as percent per fraction. (C) The same data shown in (B) now showing fold-change displacement of miR-21 per fraction for each dose level of anti-miR-21 or saline. Positive displacement values are interpreted as displacement or loss of miRNA and negative values are interpreted as enrichment or gain of miRNA, relative to let-7d reference in log₂ scale. Grey bars mark polysome containing fractions. (D) Final summary plot of average loss of miR-21 from polysome fractions for each treatment. ** p<0.01, **** p<0.0001, by one-way ANOVA with Tukey's post-hoc test. For all plots, error bars represent s.e.m. for n=6 per group.

FIG. 9. Quantification of anti-miR and miRNA in cellular lysate fractions (A) ELISA quantification of anti-miR-21 in P1 (pellet after 1,000×g spin), P16 (pellet after 16,000×g spin), and S16 (supernatant after 16,000×g spin) cellular fractions. (B) Percent miRNA in, from left to right for each sample, P1 (black bars), P16 (light grey), and S16 (dark grey) fractions. Error bars represent s.d. for n=2-3 samples per group.

FIG. 10. Comparison of miPSA with downstream target derepression in adipose tissue. (A) miPSA in adipose tissue following treatment with anti-miR-1. (B) Downstream target gene derepression scores in adipose tissue following treatment with anti-miR-1. (C) miPSA in adipose tissue following treatment with anti-miR-3. (D) Downstream target gene derepression scores in adipose tissue following treatment with anti-miR-3. *** p<0.001.

FIG. 11. Correlation of displacement value and efficacy for two different anti-miR-103/107 compounds. (A) miPSA in adipose tissue following treatment with anti-miR. (B) Downstream target gene derepression scores in adipose tissue following treatment with anti-miR. (C) OGTT in DIO mice treated with anti-miR. (D) Correlation of displacement value and efficacy for anti-miR-1. ** p<0.01, *** p<0.001, **** p<0.0001.

FIG. 12. Detection of anti-miR cross-reactivity with miPSA. Displacement value of a cross-reactive anti-miR, anti-miR-3, and a non-cross-reactive anti-miR, anti-miR-1.

FIG. 13. Measurement of mimic activity in liver and kidney for mimics of two target microRNAs. (A) miPSA in liver tissue following treatment with conjugated and unconjugated let-7a mimics. (B) miPSA in liver tissue following treatment with conjugated and unconjugated miR-34a mimics.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sangvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information. In the event that any material incorporated by reference conflicts with the express text of this specification, the express text controls.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Sample” means a composition containing, suspected of containing, or possibly containing a miRNA, an inhibitor of a miRNA, a mimic of a miRNA, and/or polysomes. Samples also include processed samples, e.g., samples subjected to lysis and/or separation procedures, such as centrifugation, e.g., density gradient centrifugation. In methods disclosed herein for determining the activity of an inhibitor of a target microRNA, a “sample” can be a composition containing, suspected of containing, or possibly containing a miRNA, an inhibitor of a miRNA, and/or polysomes. In methods disclosed herein for determining the activity of a mimic of a target microRNA, a “sample” can be a composition containing, suspected of containing, or possibly containing a miRNA, a mimic, and/or polysomes.

“Treated sample” means a sample that has been contacted with an inhibitor and/or mimic of a target microRNA. In methods disclosed herein for determining the activity of an inhibitor of a target microRNA, a “treated sample” can be a sample that has been contacted with an inhibitor of a target microRNA. In methods disclosed herein for determining the activity of a mimic of a target microRNA, a “treated sample” can be a sample that has been contacted with a mimic of a target microRNA.

“Control sample” means a sample that has not been contacted with an inhibitor or mimic of a target microRNA. A control sample may be an untreated sample, or a sample contacted with a carrier of an inhibitor or mimic of a target microRNA. In methods disclosed herein for determining the activity of an inhibitor of a target microRNA, a “control sample” can be a sample that has not been contacted with an inhibitor of a target microRNA. In methods disclosed herein for determining the activity of a mimic of a target microRNA, a “control sample” can be a sample that has not been contacted with a mimic of a target microRNA.

“Target microRNA” means a microRNA that has been selected for modulation with an inhibitory agent (e.g., in methods for determining the activity of an inhibitor of a target microRNA) or for mimicry (e.g., in methods for determining the activity of a mimic of a target microRNA).

“Mimic” means an oligomeric compound comprising an oligonucleotide comprising a nucleobase sequence that is identical to the nucleobase sequence of an endogenous microRNA and is designed to mimic the activity of the endogenous microRNA. In some embodiments, a mimic is a double-stranded compound. In some embodiments, a mimic is a single-stranded compound. In some embodiments, a mimic comprises one or more chemical modifications.

“Double-stranded compound” means a pair of oligonucleotides that are hybridized to one another or a single self-complementary oligonucleotide that forms a hairpin structure. In certain embodiments, a double-stranded compound comprises a first oligonucleotide hybridized to a second oligonucleotide. In certain embodiments, at least one of the first and second oligonucleotides is a modified oligonucleotide.

“Single-stranded compound” an oligomeric compound that is not hybridized to its complement and which lacks sufficient self-complementarity to form a stable self-duplex. In certain embodiments, a single-stranded compound is a single-stranded modified oligonucleotide.

“Polysome occupancy” means the amount of a target microRNA associated with a polysomal compartment normalized to the amount of a reference RNA associated with the same polysomal compartment.

“Polysomal compartment” means the portion of a sample that contains one or more polysomes. A polysomal compartment may be a specific portion of a fractionated sample, such as a sample fractionated on a sucrose gradient. A polysomal compartment may be a specific portion of an intact sample, such as an intact cell.

“Non-polysomal compartment” means the portion of a sample that does not contain a detectable quantity of polysomes and/or is substantially free of polysomes.

“Polysome” means complex of an mRNA molecule and two or more ribosomes that is formed during active translation. As used herein, “polysome” includes pseudo-polysomes. For a discussion of pseudo-polysomes see, e.g., Meister, G., Cell 2007; 131: 25-28.

“Absolute value” means the abundance of a molecule in a sample, expressed as individual units of the molecule in the sample. Absolute value may be the number of copies of an RNA in a sample.

“Displacement value” means the amount by which a microRNA has decreased in amount in the polysomal compartment and/or increased in amount in the non-polysomal compartment in a treated sample relative to a control sample. Displacement values can be negative, which reflects an increase in the polysomal compartment and/or decrease in amount in the non-polysomal compartment in a treated sample relative to a control sample. In some embodiments, such as embodiments involving an inhibitor of a microRNA, “displacement value” can mean the amount by which a microRNA has shifted from the polysomal to non-polysomal compartment in a treated sample relative to a control sample. A sample can be or contain, but is not limited to, a cell, a collection of cells, or a tissue.

“Accessible tissue” means a tissue of a subject from which cells can be readily removed. The cells may be removed as a tissue sample, e.g., a biopsy, or as a fluid sample, e.g., a sample of blood, plasma, saliva, urine, cerebrospinal fluid, lymph, and the like.

“Subject” means a human or non-human animal (i) selected for treatment or therapy and/or (ii) from whom a sample is obtained.

“Administering” means providing a pharmaceutical agent or composition to a subject, and includes, but is not limited to, administering by a medical professional and self-administering.

“Parenteral administration” means administration through a route other than ingestion, such as injection or infusion. Parenteral administration includes, but is not limited to, subcutaneous administration, intravenous administration, and intramuscular administration.

“Subcutaneous administration” means administration just below the skin.

“Intravenous administration” means administration into a vein.

“Therapy” means a disease treatment method. In certain embodiments, therapy includes, but is not limited to, administration of one or more pharmaceutical agents to a subject having a disease.

“Treat” means to apply one or more specific procedures used for the cure of a disease or the amelioration at least one indicator of a disease. In certain embodiments, the specific procedure is the administration of one or more pharmaceutical agents.

“Therapeutic agent” means a pharmaceutical agent used for the cure, amelioration or prevention of a disease.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Therapeutically effective amount” refers to an amount of a pharmaceutical agent that provides a therapeutic benefit to an animal.

“Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Pharmaceutically acceptable salt” means a physiologically and pharmaceutically acceptable salt of a compound provided herein, i.e., a salt that retains the desired biological activity of the compound and does not have undesired toxicological effects when administered to a subject. Nonlimiting exemplary pharmaceutically acceptable salts of compounds provided herein include sodium and potassium salt forms. The term “compound” as used herein includes pharmaceutically acceptable salts thereof unless specifically indicated otherwise.

“Anti-miR” means an oligonucleotide having a nucleobase sequence complementary to a microRNA. In certain embodiments, an anti-miR is a modified oligonucleotide.

“Anti-miR-X” where “miR-X” designates a particular microRNA, means an oligonucleotide having a nucleobase sequence complementary to miR-X. In certain embodiments, an anti-miR-X is fully complementary (i.e., 100% complementary) to miR-X. In certain embodiments, an anti-miR-X is at least 80%, at least 85%, at least 90%, or at least 95% complementary to miR-X. In certain embodiments, an anti-miR-X is a modified oligonucleotide.

“Target nucleic acid” means a nucleic acid to which an oligomeric compound is designed to hybridize.

“Targeting” means the process of design and selection of nucleobase sequence that will hybridize to a target nucleic acid.

“Targeted to” means having a nucleobase sequence that will allow hybridization to a target nucleic acid.

“Modulation” means a perturbation of function, amount, or activity. In certain embodiments, modulation means an increase in function, amount, or activity. In certain embodiments, modulation means a decrease in function, amount, or activity.

“Expression” means any functions and steps by which a gene's coded information is converted into structures present and operating in a cell.

“Nucleobase sequence” means the order of contiguous nucleobases in an oligomeric compound or nucleic acid, typically listed in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that one nucleic acid is capable of hybridizing to another nucleic acid or oligonucleotide. In certain embodiments, complementary refers to an oligonucleotide capable of hybridizing to a target nucleic acid.

“Fully complementary” means each nucleobase of an oligonucleotide is capable of pairing with a nucleobase at each corresponding position in a target nucleic acid. In certain embodiments, an oligonucleotide is fully complementary to a microRNA, i.e. each nucleobase of the oligonucleotide is complementary to a nucleobase at a corresponding position in the microRNA. A modified oligonucleotide may be fully complementary to a microRNA, and have a number of linked nucleosides that is less than the length of the microRNA. For example, an oligonucleotide with 16 linked nucleosides, where each nucleobase of the oligonucleotide is complementary to a nucleobase at a corresponding position in a microRNA, is fully complementary to the microRNA. In certain embodiments, an oligonucleotide wherein each nucleobase has complementarity to a nucleobase within a region of a microRNA stem-loop sequence is fully complementary to the microRNA stem-loop sequence.

“Percent complementarity” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Percent identity” means the number of nucleobases in a first nucleic acid that are identical to nucleobases at corresponding positions in a second nucleic acid, divided by the total number of nucleobases in the first nucleic acid. In certain embodiments, the first nucleic acid is a microRNA and the second nucleic acid is a microRNA. In certain embodiments, the first nucleic acid is an oligonucleotide and the second nucleic acid is an oligonucleotide.

“Hybridize” means the annealing of complementary nucleic acids that occurs through nucleobase complementarity.

“Mismatch” means a nucleobase of a first nucleic acid that is not capable of Watson-Crick pairing with a nucleobase at a corresponding position of a second nucleic acid.

“Identical” in the context of nucleobase sequences, means having the same nucleobase sequence, independent of sugar, linkage, and/or nucleobase modifications and independent of the methyl state of any pyrimidines present.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (http://microma.sanger.ac.uk/). In certain embodiments, microRNA is abbreviated as “microRNA” or “miR.”

“Pre-microRNA” or “pre-miR” means a non-coding RNA having a hairpin structure, which is the product of cleavage of a pri-miR by the double-stranded RNA-specific ribonuclease known as Drosha.

“Stem-loop sequence” means an RNA having a hairpin structure and containing a mature microRNA sequence. Pre-microRNA sequences and stem-loop sequences may overlap. Examples of stem-loop sequences are found in the microRNA database known as miRBase (http://microma.sanger.ac.uk/).

“Pri-microRNA” or “pri-miR” means a non-coding RNA having a hairpin structure that is a substrate for the double-stranded RNA-specific ribonuclease Drosha.

“microRNA precursor” means a transcript that originates from a genomic DNA and that comprises a non-coding, structured RNA comprising one or more microRNA sequences. For example, in certain embodiments a microRNA precursor is a pre-microRNA. In certain embodiments, a microRNA precursor is a pri-microRNA.

“microRNA-regulated transcript” means a transcript that is regulated by a microRNA.

“Seed sequence” means a nucleobase sequence comprising from 6 to 8 contiguous nucleobases of nucleobases 1 to 9 of the 5′-end of a mature microRNA sequence.

“Seed match sequence” means a nucleobase sequence that is complementary to a seed sequence, and is the same length as the seed sequence.

“Oligomeric compound” means a compound that comprises a plurality of linked monomeric subunits. Oligomeric compounds include oligonucleotides.

“Oligonucleotide” means a compound comprising a plurality of linked nucleosides, each of which can be modified or unmodified, independent from one another.

“Naturally occurring internucleoside linkage” means a 3′ to 5′ phosphodiester linkage between nucleosides.

“Natural sugar” means a sugar found in DNA (2′-H) or RNA (2′-OH).

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

“Compound comprising a modified oligonucleotide consisting of” a number of linked nucleosides means a compound that includes a modified oligonucleotide having the specified number of linked nucleosides. Thus, the compound may include additional substituents or conjugates. Unless otherwise indicated, the compound does not include any additional nucleosides beyond those of the modified oligonucleotide. For example, unless otherwise indicated, a compound comprising a modified oligonucleotide does not include a complementary strand hybridized to the modified oligonucleotide (i.e., the modified oligonucleotide is a single-stranded modified oligonucleotide).

“Modified oligonucleotide” means an oligonucleotide having one or more modifications relative to a naturally occurring terminus, sugar, nucleobase, and/or internucleoside linkage. A modified oligonucleotide may comprise unmodified nucleosides.

“Single-stranded modified oligonucleotide” means a modified oligonucleotide which is not hybridized to a complementary strand.

“Modified nucleoside” means a nucleoside having any change from a naturally occurring nucleoside. A modified nucleoside may have a modified sugar and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

“Modified internucleoside linkage” means any change from a naturally occurring internucleoside linkage.

“Phosphorothioate internucleoside linkage” means a linkage between nucleosides where one of the non-bridging atoms is a sulfur atom.

“Modified sugar moiety” means substitution and/or any change from a natural sugar.

“Unmodified nucleobase” means the naturally occurring heterocyclic bases of RNA or DNA: the purine bases adenine (A) and guanine (G), and the pyrimidine bases thymine (T), cytosine (C) (including 5-methylcytosine), and uracil (U).

“5-methylcytosine” means a cytosine comprising a methyl group attached to the 5 position.

“Non-methylated cytosine” means a cytosine that does not have a methyl group attached to the 5 position.

“Modified nucleobase” means any nucleobase that is not an unmodified nucleobase.

“Sugar moiety” means a naturally occurring furanosyl or a modified sugar moiety.

“Modified sugar moiety” means a substituted sugar moiety or a sugar surrogate.

“2′-O-methyl sugar” or “2′-OMe sugar” means a sugar having an O-methyl modification at the 2′ position.

“2′-O-methoxyethyl sugar” or “2′-MOE sugar” means a sugar having an O-methoxyethyl modification at the 2′ position.

“2′-fluoro” or “2′-F” means a sugar having a fluoro modification of the 2′ position.

“Bicyclic sugar moiety” means a modified sugar moiety comprising a 4 to 7 membered ring (including by not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl. Nonlimiting exemplary bicyclic sugar moieties include LNA, ENA, cEt, S-cEt, and R-cEt.

“Locked nucleic acid (LNA) sugar moiety” means a substituted sugar moiety comprising a (CH₂)—O bridge between the 4′ and 2′ furanose ring atoms.

“ENA sugar moiety” means a substituted sugar moiety comprising a (CH₂)₂—O bridge between the 4′ and 2′ furanose ring atoms.

“Constrained ethyl (cEt) sugar moiety” means a substituted sugar moiety comprising a CH(CH₃)—O bridge between the 4′ and the 2′ furanose ring atoms. In certain embodiments, the CH(CH₃)—O bridge is constrained in the S orientation. In certain embodiments, the (CH₂)₂—O is constrained in the R orientation.

“S-cEt sugar moiety” means a substituted sugar moiety comprising an S-constrained CH(CH₃)—O bridge between the 4′ and the 2′ furanose ring atoms.

“R-cEt sugar moiety” means a substituted sugar moiety comprising an R-constrained CH(CH₃)—O bridge between the 4′ and the 2′ furanose ring atoms.

“2′-O-methyl nucleoside” means a 2′-modified nucleoside having a 2′-O-methyl sugar modification.

“2′-O-methoxyethyl nucleoside” means a 2′-modified nucleoside having a 2′-O-methoxyethyl sugar modification. A 2′-O-methoxyethyl nucleoside may comprise a modified or unmodified nucleobase.

“2′-fluoro nucleoside” means a 2′-modified nucleoside having a 2′-fluoro sugar modification. A 2′-fluoro nucleoside may comprise a modified or unmodified nucleobase.

“Bicyclic nucleoside” means a 2′-modified nucleoside having a bicyclic sugar moiety. A bicyclic nucleoside may have a modified or unmodified nucleobase.

“cEt nucleoside” means a nucleoside comprising a cEt sugar moiety. A cEt nucleoside may comprise a modified or unmodified nucleobase.

“S-cEt nucleoside” means a nucleoside comprising an S-cEt sugar moiety.

“R-cEt nucleoside” means a nucleoside comprising an R-cEt sugar moiety.

“β-D-deoxyribonucleoside” means a naturally occurring DNA nucleoside.

“β-D-ribonucleoside” means a naturally occurring RNA nucleoside.

“LNA nucleoside” means a nucleoside comprising a LNA sugar moiety.

“ENA nucleoside” means a nucleoside comprising an ENA sugar moiety.

“Approximately” means, unless context indicates otherwise, a value (e.g., numerical value) plus or minus 10% (e.g., +/−9%, +/−8%, +/−7%, +/−6%, +/−5%, +/−4%, +/−3%, +/−2%, +/−1%, or other non-integer values encompassed therein).

Overview

The drug development process frequently involves determining the extent of drug-target engagement. In the case of modulators of microRNAs, such as microRNA inhibitors, common approaches for assessing drug-target engagement include determining the amount of detectable microRNA remaining after anti-miR treatment, and determining the derepression of downstream microRNA-regulated genes. While widely used, the biological and technical challenges associated with these methods may complicate drug discovery decisions such as target validation and lead compound selection.

Provided here are methods for direct measurement of microRNA engagement by a microRNA inhibitor or mimic, which can provide more robust performance than conventional pharmacodynamics using downstream target gene derepression. In general, active microRNAs (endogenous microRNAs or mimics) bind target mRNAs in high molecular weight polysome complexes, while inhibited microRNAs are sterically blocked from forming this interaction. Accordingly, in methods of this disclosure involving a sample treated with an inhibitor of a miRNA, the amounts of a miRNA in polysomal and non-polysomal compartments may considered indicative of the level of activity or inhibition, respectively, of the miRNA. Treatment with a microRNA inhibitor can cause a specific shift, or displacement, of cognate microRNA from polysomal to non-polysomal compartments of a sample. On the other hand, a mimic can also interact with high molecular weight polysome complexes along with the microRNA and be detected in place of or in combination with the microRNA. Thus, in methods of this disclosure involving a sample treated with a mimic, the amounts of a miRNA (including the mimic) in polysomal and non-polysomal compartments may considered indicative of the level of activity of the mimic. Treatment with a mimic can cause an enrichment, or negative displacement, of the microRNA in polysomal relative to non-polysomal compartments of a sample. Regardless of whether a mimic or an inhibitor is used, the magnitude of the displacement value observed for a sample can be dose-responsive and can maintain a linear relationship with downstream target gene derepression while providing a substantially higher dynamic window for assessing inhibitory activity. These methods permit the robust and reliable measurement of the extent to which a microRNA inhibitor engages with its target microRNA or a mimic engages with its target RNA.

The methods provided herein may be used to measure the activity of a microRNA inhibitor or mimic, independently of whether the downstream genes regulated by the microRNA are known, or are reliable measures of inhibitor or mimic activity. Further, the methods provided herein permit the measurement of the activity of a microRNA inhibitor or mimic in a cell, tissue or organ, in a subject treated with a microRNA inhibitor or mimic, including a cell, tissue or organ that may not be the primary site of action of the microRNA inhibitor or mimic, but is more readily accessible than the primary site of action.

Certain Uses of the Invention

Provided here are methods for determining the activity of an inhibitor or mimic of a target microRNA in a sample that has been contacted with the inhibitor or mimic of the target miRNA. In certain embodiments, the sample is a single cell. In certain embodiments, the sample is a collection of cells. In certain embodiments, the sample is a tissue. In certain embodiments, the contacting occurs in vitro. In certain embodiments, the contacting occurs in vivo.

In some embodiments, a sample is prepared by removing cells from an organism. In some embodiments, the cells are removed as a tissue sample, e.g., a biopsy. In some embodiments, the cells are removed as a fluid sample, e.g., a sample of blood, plasma, saliva, urine, cerebrospinal fluid, lymph, and the like. In some embodiments, the cells are lysed. In some embodiments, the lysate is separated to give one or more fractions containing polysomes. Exemplary forms of separation include centrifugation, including density gradient centrifugation. Exemplary forms of separation also include chromatography, such as size exclusion chromatography. In some embodiments, the lysate is separated to give one or more fractions substantially free of polysomes. A fraction may be considered substantially free of polysomes where a technique for detecting polysomes does not detect polysomes in an amount statistically significantly above the background level of the technique.

Treatment with a microRNA inhibitor or mimic can cause a specific shift, or displacement, of cognate microRNA between polysomal and non-polysomal compartments of a sample. The level of a microRNA can be determined in each of the polysomal and non-polysomal compartments, permitting the determination of the polysome occupancy for the target microRNA, in each of the treated and control samples. Accordingly, the methods provided herein comprise determining a polysome occupancy in a treated sample, determining a polysome occupancy in a control sample, and comparing the polysome occupancy in the treated sample to the polysome occupancy in the control sample to determine a displacement value for the inhibitor or mimic. When an inhibitor is used, the displacement value can represent the shift of the microRNA between the polysome complex where the microRNA is active and the non-polysomal compartment where the microRNA is inhibited, and provides a direct measurement of the activity of the microRNA inhibitor. When a mimic is used, the displacement value represents the changes relative to the control sample in level of the microRNA (including the mimic in the treated sample) in the polysome complex where the microRNA and mimic are active and in the non-polysomal compartment, and provides a direct measurement of the activity of the mimic. In general, an inhibitor is expected to promote a shift of a microRNA from the polysome complex where the microRNA is active to the non-polysomal compartment where the microRNA is inhibited. It is also possible, e.g. when complex regulatory interactions occur, such as where an inhibitor inhibits a first microRNA which directly or indirectly inhibits a second miRNA or where a mimic directly or indirectly disinhibits (e.g., inhibits an inhibitor of) the second miRNA, for the inhibitor or mimic to promote a shift of a microRNA in the other direction: from the non-polysomal compartment where the microRNA is inhibited to the polysome complex where the microRNA is active. In general, a mimic is expected to give a negative displacement value, e.g., reflecting an increase in the level of the microRNA (including the mimic) in the polysomal compartment where the microRNA is active.

Displacement values may be calculated based on microRNA levels measured by a variety of methods. Measurement of microRNA levels can refer to absolute levels (e.g., numbers of copies of a specific microRNA per sample, or a concentration of the miRNA in the sample) or relative levels (i.e., a control sample has twice as much microRNA as a treated sample).

A common method for relative quantitation of microRNA levels is the ΔΔC_{T} method. C_{T}, (threshold cycle) is the number of cycles that it takes for a real-time amplification reaction to cross the fluorescence threshold, a fluorescent signal significantly above the background fluorescence for the reaction. C_{T} values are a relative measure of the concentration of target RNA in an amplification reaction. For each of the control and treated samples, the difference in C_{T} values for the target microRNA and the reference RNA is calculated (the ΔC_{T}). Next, subtraction of the control sample ΔC_{T} from the treated sample ΔC_{T} yields the ΔΔC_{T}. An example calculation is provided in Table A.

TABLE A
ΔΔC_{T} Method to Calculate Displacement Value

| | | Polysome Occupancy |
|---|---|---|
| C_{T} Value | C_{T} Value | Treated Sample |
| Target microRNA | Reference RNA | (Treated ΔC_{T}) |
| Treated Sample | Treated Sample | (Target C_{T} − Control C_{T}) |
| 13.32 | 9.15 | 4.17 |
| | | Polysome Occupancy |
| C_{T} Value | C_{T} Value | Control Sample |
| Target microRNA | Reference RNA | (Control ΔC_{T}) |
| Control Sample | Control Sample | (Target C_{T} − Control C_{T}) |
| 10 | 9 | 1 |

| | |
|---|---|
| Displacement Value (ΔΔC_{T}) | 3.17 |
| (Treated ΔC_{T} − Control ΔC_{T}) | |

When using a microRNA quantitation method that provides absolute values of microRNA, the displacement value is calculated as demonstrated in Table B.

TABLE B
Calculating Displacement Value with Absolute microRNA Levels

| Absolute Value | Absolute Value | Polysome Occupancy |
|---|---|---|
| Target microRNA | Reference RNA | Treated Sample |
| Treated Sample | Treated Sample | (Target Count/Reference Count) |
| 50 | 900 | 0.06 |
| Absolute Value | Absolute Value | Polysome Occupancy |
| Target microRNA | Reference RNA | Control Sample |
| Control Sample | Control Sample | (Target Count/Reference Count) |
| 500 | 1000 | .05 |

| | |
|---|---|
| Displacement Value = | 3.17 |
| log₂(Polysome Occupancy Control/ | |
| Polysome Occupancy Treated) | |

In certain embodiments, a displacement value is represented by the logarithm of the treated sample polysome occupancy less the logarithm of the control sample polysome occupancy. For example, the displacement value can be represented by the following formula in which polysome occupancies are expressed as logarithmic values:

D=[polysome occupancy,treated sample]−[polysome occupancy,control sample]

In certain embodiments, a displacement value is represented by the logarithm of the quotient of control sample polysome occupancy divided by treated sample polysome occupancy. For example, the displacement value can be represented by the following formula in which polysome occupancies are expressed as absolute values:

$$D = {log}_{2} ( \frac{polysome occupancy , control sample}{polysome occupancy , treated sample} )$$

In certain embodiments, determining the polysome occupancy of the target miRNA in the treated sample comprises:
i. measuring the amount of the target microRNA in the treated sample by quantitative PCR to generate a target microRNA Ct value for the treated sample;
ii. measuring the amount of a reference microRNA in the treated sample by quantitative PCR to generate a reference RNA Ct value for the treated sample; and
iii. subtracting the reference RNA Ct value for the treated sample from the target microRNA Ct value for the treated sample, wherein the resulting value is the polysome occupancy for the treated sample; and
determining the polysome occupancy of the target microRNA in the control sample comprises:
i. measuring the amount of the target microRNA in the control sample by quantitative PCR to generate a target microRNA Ct value for the control sample;
ii. measuring the amount of a reference RNA in the control sample by quantitative PCR to generate a reference RNA Ct value for the control sample; and
iii. subtracting the reference RNA Ct value for the control sample from the target microRNA Ct value for the control sample, wherein the resulting value is the polysome occupancy for the control sample.

In certain embodiments, determining the displacement value for the inhibitor or mimic of the target microRNA comprises subtracting the polysome occupancy of the control sample from the poly some occupancy of the treated sample, wherein the resulting value is the displacement value for the target microRNA.

In certain embodiments, the polysome occupancy is the amount of a target microRNA associated with a polysomal compartment normalized to the amount of a reference RNA associated with the same polysomal compartment.

The methods provided herein can take advantage of one or more different characteristics of polysomal (active microRNA) and non-polysomal (inhibited or inactive microRNA) compartments of a sample, such as a cell.

In certain embodiments, the methods provided herein utilize the size differences between polysomal and non-polysomal compartments of a cell, and the polysomal compartment of a sample is isolated by differential ultracentrifugation through a sucrose gradient. In certain embodiments, a sample is layered on top of a sucrose gradient and spun in an ultracentrifuge. In some embodiments, the sucrose gradient comprises sucrose concentrations ranging from 20%-40%. In some embodiments, the sucrose gradient comprises sucrose concentrations ranging from 15%-45%. In some embodiments, the sucrose gradient comprises sucrose concentrations ranging from 10%-50%. In some embodiments, the sucrose gradient comprises sucrose concentrations ranging from 10%-55%. In some embodiments, the sucrose gradient comprises sucrose concentrations ranging from 15%-60%. In some embodiments, the sucrose gradient comprises sucrose concentrations ranging from 5%-50%. In some embodiments, the sucrose gradient comprises sucrose concentrations ranging from 5%-60%. As used herein, a gradient comprises a range of concentrations if the gradient includes those concentrations at any point within the gradient. Thus, for example, a 5-60% gradient comprises a 20%-40% gradient. In some embodiments, the sucrose gradient is approximately 5-60%. In some embodiments, the sucrose gradient is 5-60%. In certain embodiments, the ultracentrifuge is operated at approximately 30,000-50,000 rpm, 35,000-45,000 rpm, or 40,000 rpm. In certain embodiments, the ultracentrifuge is operated for approximately 0.5-3, 1-2, 1.25-1.75, 1.4-1.6, 1.45-1.55, or 1.5 hours.

In certain embodiments, the polysomal compartment of a sample is isolated by size-exclusion chromatography.

In some embodiments, one or more polysomal compartments of the separated lysate are identified. Identification can be performed based on the characteristics of a fraction evident from the separation procedure such as sedimentation coefficient in the case of centrifugation or apparent molecular weight or elution time in the case of size exclusion chromatography. Alternatively, identification can be performed based on data independent of or in addition to data from the separation procedure, such as detection of rRNA, ribosomal proteins, complexed rRNA and mRNA, and the like. In some embodiments, identification comprises measuring the amount of an RNA in at least one polysomal compartment and measuring the amount of an RNA in at least one non-polysomal compartment.

Procedures for quantification or measurement of RNA that can be used include quantitative PCR, spectrophotometry, electrophoresis, hybridization, precipitation, fluorometry, colorimetry, densitometry, scintillation counting, autoradiography, or a combination of two or more of the foregoing procedures. In certain embodiments, the method comprises measuring the amount of an RNA by a procedure comprising contacting the RNA with a detectably labeled oligonucleotide to form a detection complex and detecting the detection complex. In certain embodiments, the method comprises measuring the amount of an RNA by a procedure comprising contacting the RNA with a primer, performing a nucleic acid synthesis reaction in which the primer is extended, and detecting nucleic acid produced by the nucleic acid synthesis reaction. The nucleic acid synthesis reaction can be an amplification reaction, such as PCR (including RT-PCR), such as real-time and/or quantitative RT-PCR.

In any of the embodiments described herein, the RNA is a target microRNA. In any of the embodiments described herein, the RNA is a reference RNA.

In certain embodiments, the reference RNA is a small non-coding RNA. In certain embodiments, the reference RNA is a messenger RNA. In certain embodiments, the reference RNA is a long non-coding RNA. In certain embodiments, the reference RNA is a microRNA. In certain embodiments, the reference RNA is a member of the let-7 family. In certain embodiments, the reference RNA is let-7d.

In certain embodiments, the treated sample is a single cell. In certain embodiments, the treated sample is a collection of cells. In certain embodiments, the cells are from healthy tissue. In certain embodiments, the cells are from a diseased tissue. In some embodiments, sample comprises a neoplastic, hyperplastic, dysplastic, or metaplastic cell. In some embodiments, the sample comprises a benign hyperplastic, dysplastic, or metaplastic cell. In some embodiments, the sample comprises a malignant cell. In some embodiments, the sample comprises a somatic cell. In some embodiments, the sample comprises an epithelial cell. In some embodiments, the sample comprises an endothelial cell. In some embodiments, the sample comprises a leukocyte. In some embodiments, the sample comprises one or more of neutrophils, lymphocytes, monocytes, eosinophils, basophils, adipocytes, chondrocytes, pancreatic islet cells, thyroid cells, parathyroid cells, parotid cells, neuronal cells, astrocytes, glial cells, macrophages, epithelial cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, and osteoclasts.

In certain embodiments, a sample is collected from a subject treated with an inhibitor or mimic of a target microRNA. In certain embodiments, the subject is treated with a modified oligonucleotide targeted to a microRNA. In certain embodiments, the subject is treated with a modified oligonucleotide that is a mimic. In certain embodiments, the subject is a human subject.

In a subject treated with a microRNA inhibitor or mimic, measurement of the extent to which a microRNA inhibitor engages with its target microRNA or a mimic engages with its target RNA in certain cell or tissue types may be complicated by the difficulty, and potential risk to the health of the subject, of obtaining a sample from such cell or tissue types. In such cases, surrogate markers of microRNA inhibition or displacement may be used to assess the activity of the microRNA inhibitor or mimic.

For certain microRNAs, downstream markers of microRNA inhibition are available and can serve as surrogates for the inhibition of a particular microRNA. For example, as inhibition of the liver-specific microRNA miR-122 results in the lowering of cholesterol levels in the blood of a subject, blood cholesterol levels of the subject provide an indirect measurement of the miR-122 inhibitor. A biopsy of the liver is not needed to determine the activity of a miR-122 inhibitor. For many microRNAs, however, surrogate markers of activity are not readily available, or are not reproducible and reliable, or obtaining a sample of certain cells or tissues is not practical. For example, the microRNA miR-21 plays a role in kidney disease. While it may be desirable to determine the extent of miR-21 inhibition in the kidney in the context of disease, collecting a biopsy from a diseased kidney may not be practical or safe to a subject. Additionally, as described herein, the derepression of miR-21 downstream target genes is not a robust measurement of miR-21 inhibition, thus even if a sample of a kidney could be obtained, target gene derepression is not a reliable indicator of miR-21 inhibition. Similarly, the repression of miR-21 downstream target genes may not be a robust measurement of miR-21 mimic activity, thus even if a sample of a kidney could be obtained, target gene repression is not a reliable indicator of miR-21 mimic activity. Accordingly, additional methods are needed to measure the activity of a microRNA inhibitor or mimic in cases where downstream target derepression may not provide sufficient information, and/or in an accessible tissue of a subject treated with the microRNA inhibitor or mimic.

As demonstrated herein, the miPSA is capable of measuring microRNA inhibition where downstream microRNA target derepression may be weak, or where microRNA downstream targets may not be known. The miPSA is also capable of measuring mimic activity where downstream target repression by the mimic may be weak, or where microRNA downstream targets may not be known. Additionally, the miPSA is capable of measuring microRNA inhibition or mimic activity in an accessible tissue, including but not limited to adipose tissue, where the displacement value for the microRNA inhibitor or mimic in the accessible tissue is representative of the displacement value in other cells or tissues where the microRNA inhibitor or mimic is active. Accordingly, the methods provided herein may be used to measure microRNA inhibitor or mimic activity, independently of the behavior of downstream target genes of the microRNA, in a sample prepared from a tissue that may be conveniently biopsied. In any of the embodiments provided herein, a sample is prepared from an accessible tissue. In certain embodiments, the accessible tissue is prepared from adipose tissue. In certain embodiments, each of the treated sample and control sample are prepared from adipose tissue. In certain embodiments, the accessible tissue is blood. In certain embodiments, each of the treated sample and control sample are prepared from blood.

In any of the embodiments provided herein, such as embodiments involving a microRNA inhibitor, at least one downstream target of the microRNA is not measurably derepressed in the treated sample. In any of the embodiments provided herein, such as embodiments involving a microRNA inhibitor, at least two, at least three, at least four, or at least five downstream targets of the microRNA are not measurably derepressed in the treated sample. In any of the embodiments provided herein, such as embodiments involving a mimic, at least one downstream target of the microRNA is not measurably repressed in the treated sample. In any of the embodiments provided herein, such as embodiments involving a mimic, at least two, at least three, at least four, or at least five downstream targets of the microRNA are not measurably repressed in the treated sample.

In any of the embodiments provided herein, such as embodiments involving a microRNA inhibitor, at least one target of the microRNA has previously been determined not to be measurably derepressed following treatment with the same inhibitor of the target microRNA. In any of the embodiments provided herein, such as embodiments involving a microRNA inhibitor, at least two, at least three, at least four, or at least five downstream targets of the microRNA have previously been determined not to be measurably derepressed following treatment with the same inhibitor of the target microRNA. In any of the embodiments provided herein, such as embodiments involving a mimic, at least one target of the microRNA has previously been determined not to be measurably repressed following treatment with the same mimic of the target microRNA. In any of the embodiments provided herein, such as embodiments involving a mimic, at least two, at least three, at least four, or at least five downstream targets of the microRNA have previously been determined not to be measurably repressed following treatment with the same mimic of the target microRNA. In any of the embodiments provided herein, the downstream target of the microRNA is a messenger RNA.

In any of the embodiments provided herein, the target microRNA is miR-103. In any of the embodiments provided herein, the target microRNA is miR-107. In any of the embodiments provided herein, the target microRNA is miR-103 and miR-107. In any of the embodiments provided herein, the target microRNA is miR-21. In any of the embodiments provided herein, the target microRNA is miR-122. In any of the embodiments provided herein, the target microRNA is miR-17.

In certain embodiments, the inhibitor of the microRNA is a modified oligonucleotide, wherein the nucleobase sequence of the modified oligonucleotide is complementary to the target microRNA. In certain embodiments, the modified oligonucleotide is complementary to a single microRNA. In certain embodiments, the modified oligonucleotide is complementary to a family of microRNAs.

In certain embodiments, the inhibitor of the microRNA is a small molecule.

In certain embodiments, the mimic is a double-stranded compound. In certain embodiments, the mimic is a single-stranded compound.

Certain MicroRNA Nucleobase Sequences

In certain embodiments, the invention involves mimics comprising an oligonucleotide having a nucleobase sequence with identity to the nucleobase sequence of a microRNA. In certain embodiments, the invention involves mimics comprising a modified oligonucleotide having a nucleobase sequence with identity to the nucleobase sequence of a microRNA. Nucleobase sequences of mature miRNAs and their corresponding stem-loop sequences described herein are the sequences found in miRBase, an online searchable database of miRNA sequences and annotation, found at the website microRNA “dot” sanger “dot” ac “dot” uk.

In certain embodiments, a mimic comprises an oligonucleotide having a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the microRNA.

In certain embodiments, the mimic is a double-stranded compound comprising a first oligonucleotide hybridized to a second oligonucleotide, wherein the first oligonucleotide comprises a nucleobase sequence having identity to the nucleobase sequence of a microRNA, and the second oligonucleotide comprises a nucleobase sequence that is complementary to the nucleobase sequence of the first oligonucleotide. In certain embodiments, the first oligonucleotide of a double-stranded compound is a modified oligonucleotide. In certain embodiments, the second oligonucleotide of a double-stranded compound is a modified oligonucleotide. In certain embodiments, each of the first and second oligonucleotides is a modified oligonucleotide.

In certain embodiments, the second oligonucleotide is 100% complementary to the first oligonucleotide. In certain embodiments, the second oligonucleotide comprises one or more mismatches with respect to the first oligonucleotide.

In certain embodiments, the hybridization of a first oligonucleotide to a second oligonucleotide forms at least one blunt end. In certain such embodiments, the hybridization of a first oligonucleotide to a second oligonucleotide forms a blunt end at each terminus of the double-stranded compound.

The hybridization of a first oligonucleotide to a second oligonucleotide may result in the formation of one or more overhangs, where one or more additional nucleosides of at least one terminus of the first oligonucleotide do not have a corresponding nucleobase in the second oligonucleotide with which to pair through hydrogen bonding. In such cases, the hybridization of the first oligonucleotide to the second oligonucleotide results in the formation of a central complementary region. The central complementary region can tolerate mismatches, provided that there is sufficient complementarity to permit hybridization. In certain embodiments, there are 0, 1, 2, or 3 mismatches in the central complementary region.

In certain embodiments, a terminus of a first oligonucleotide comprises one or more additional linked nucleosides relative to the number of linked nucleosides of the second oligonucleotide. In certain embodiments, the one or more additional nucleosides are at the 5′ terminus of the first or second oligonucleotide. In certain embodiments, the one or more additional nucleosides are at the 3′ terminus of the first or second oligonucleotide. In certain embodiments, two additional linked nucleosides are linked to a terminus. In certain embodiments, one additional nucleoside is linked to a terminus.

In certain embodiments, a mimic that is a double-stranded compound comprises one or more conjugate moieties. In certain embodiments, a first oligonucleotide is linked to a conjugate moiety at the 5′ terminus. In certain embodiments, a first oligonucleotide is linked to a conjugate moiety at the 3′ terminus. In certain embodiments, a second oligonucleotide is linked to a conjugate moiety at the 5′ terminus. In certain embodiments, a second oligonucleotide is linked to a conjugate moiety at the 3′ terminus.

In certain embodiments, the mimic is a single-stranded compound, wherein the single-stranded compound comprises an oligonucleotide comprising a nucleobase sequence having identity to the nucleobase sequence of a microRNA. In certain embodiments, the oligonucleotide of the single-stranded compound is a modified oligonucleotide. In certain embodiments, the oligonucleotide is linked to one or more conjugate moieties. In certain embodiments, an oligonucleotide or modified oligonucleotide of a mimic is linked to a conjugate moiety at the 5′ terminus and/or at the 3′ terminus.

In some embodiments, the invention involves modified oligonucleotides having a nucleobase sequence that is complementary to the nucleobase sequence of a microRNA, or a precursor thereof. In certain embodiments, each nucleobase of the modified oligonucleotide is capable of undergoing base-pairing with a nucleobase at each corresponding position in the nucleobase sequence of a microRNA, or a precursor thereof. In certain embodiments the nucleobase sequence of a modified oligonucleotide may have one or more mismatched base pairs with respect to the nucleobase sequence of a microRNA or precursor sequence, and remains capable of hybridizing to its target sequence.

In certain embodiments, an oligonucleotide or modified oligonucleotide consists of a number of linked nucleosides that is equal to the length of a microRNA. In certain embodiments, an oligonucleotide or modified oligonucleotide consists of a number of linked nucleosides that is equal to the length of a microRNA plus or minus one nucleoside. In certain embodiments, an oligonucleotide or modified oligonucleotide consists of a number of linked nucleosides that is equal to the length of a microRNA plus or minus two nucleosides. In certain embodiments, an oligonucleotide or modified oligonucleotide consists of a number of linked nucleosides that is equal to the length of a microRNA plus or minus three nucleosides.

In certain embodiments, the number of linked nucleosides of an oligonucleotide or modified oligonucleotide is less than the length of microRNA. An oligonucleotide or modified oligonucleotide having a number of linked nucleosides that is less than the length of the microRNA, wherein each nucleobase of the oligonucleotide or modified oligonucleotide is complementary to each nucleobase at a corresponding position of the microRNA, is considered to be an oligonucleotide or modified oligonucleotide having a nucleobase sequence that is fully complementary to a region of the microRNA sequence. An oligonucleotide or modified oligonucleotide having a number of linked nucleosides that is less than the length of the microRNA, wherein each nucleobase of the oligonucleotide or modified oligonucleotide is identical to each nucleobase at a corresponding position of the microRNA, is considered to be an oligonucleotide or modified oligonucleotide having a nucleobase sequence that is fully identical to a region of the microRNA sequence. For example, an oligonucleotide or modified oligonucleotide consisting of 19 linked nucleosides, where either each nucleobase is complementary or each nucleobase is identical to a corresponding position of a microRNA that is 22 nucleobases in length, is fully complementary or fully identical to a 19 nucleobase region of a microRNA. Such an oligonucleotide or modified oligonucleotide has 100% complementarity or identity to (or is fully complementary or identical to) a 19 nucleobase segment of the microRNA, and is considered to be 100% complementary or identical to (or fully complementary or identical to) the microRNA.

In certain embodiments, an oligonucleotide or modified oligonucleotide comprises a nucleobase sequence that is complementary to a seed sequence, i.e. an oligonucleotide or modified oligonucleotide comprises a seed-match sequence. In certain embodiments, an oligonucleotide or modified oligonucleotide comprises a nucleobase sequence that is identical to a seed sequence. In certain embodiments, a seed sequence is a hexamer seed sequence. In certain such embodiments, a seed sequence is nucleobases 1-6 of a microRNA. In certain such embodiments, a seed sequence is nucleobases 2-7 of a microRNA. In certain such embodiments, a seed sequence is nucleobases 3-8 of a microRNA. In certain embodiments, a seed sequence is a heptamer seed sequence. In certain such embodiments, a heptamer seed sequence is nucleobases 1-7 of a microRNA. In certain such embodiments, a heptamer seed sequence is nucleobases 2-8 of a microRNA. In certain embodiments, the seed sequence is an octamer seed sequence. In certain such embodiments, an octamer seed sequence is nucleobases 1-8 of a microRNA. In certain embodiments, an octamer seed sequence is nucleobases 2-9 of a microRNA.

In certain embodiments, an oligonucleotide or modified oligonucleotide has a nucleobase sequence having one mismatch with respect to the nucleobase sequence of a microRNA, or a precursor thereof. In certain embodiments, an oligonucleotide or modified oligonucleotide has a nucleobase sequence having two mismatches with respect to the nucleobase sequence of a microRNA, or a precursor thereof. In certain such embodiments, an oligonucleotide or modified oligonucleotide has a nucleobase sequence having no more than two mismatches with respect to the nucleobase sequence of a microRNA, or a precursor thereof. In certain such embodiments, the mismatched nucleobases are contiguous. In certain such embodiments, the mismatched nucleobases are not contiguous.

Certain Oligonucleotides and Modified Oligonucleotides

In certain embodiments, an oligonucleotide or modified oligonucleotide consists of 8 to 25 linked nucleosides. In certain embodiments, an oligonucleotide or modified oligonucleotide consists of 8 to 12 linked nucleosides. In certain embodiments, an oligonucleotide or modified oligonucleotide consists of 12 to 25 linked nucleosides. In certain embodiments, an oligonucleotide or modified oligonucleotide consists of 15 to 25 linked nucleosides. In certain embodiments, an oligonucleotide or modified oligonucleotide consists of 15 to 19 linked nucleosides. In certain embodiments, an oligonucleotide or modified oligonucleotide consists of 15 to 16 linked nucleosides. In certain embodiments, an oligonucleotide or modified oligonucleotide consists of 17 to 23 linked nucleosides. In certain embodiments, an oligonucleotide or modified oligonucleotide consists of 19 to 23 linked nucleosides.

In certain embodiments, an oligonucleotide or modified oligonucleotide consists of 8 linked nucleosides. In certain embodiments, an oligonucleotide or modified oligonucleotide consists of 9 linked nucleosides. In certain embodiments, an oligonucleotide or modified oligonucleotide consists of 10 linked nucleosides. In certain embodiments, an oligonucleotide or modified oligonucleotide consists of 11 linked nucleosides. In certain embodiments, an oligonucleotide or modified oligonucleotide consists of 12 linked nucleosides. In certain embodiments, an oligonucleotide or modified oligonucleotide consists of 13 linked nucleosides. In certain embodiments, an oligonucleotide or modified oligonucleotide consists of 14 linked nucleosides. In certain embodiments, an oligonucleotide or modified oligonucleotide consists of 15 linked nucleosides. In certain embodiments, an oligonucleotide or modified oligonucleotide consists of 16 linked nucleosides. In certain embodiments, an oligonucleotide or modified oligonucleotide consists of 17 linked nucleosides. In certain embodiments, an oligonucleotide or modified oligonucleotide consists of 18 linked nucleosides. In certain embodiments, an oligonucleotide or modified oligonucleotide consists of 19 linked nucleosides. In certain embodiments, an oligonucleotide or modified oligonucleotide consists of 20 linked nucleosides.

In certain embodiments, an oligonucleotide or modified oligonucleotide consists of 21 linked nucleosides. In certain embodiments, an oligonucleotide or modified oligonucleotide consists of 22 linked nucleosides. In certain embodiments, an oligonucleotide or modified oligonucleotide consists of 23 linked nucleosides. In certain embodiments, an oligonucleotide or modified oligonucleotide consists of 24 linked nucleosides. In certain embodiments, an oligonucleotide or modified oligonucleotide consists of 25 linked nucleosides.

In certain embodiments, an oligonucleotide or modified oligonucleotide comprises one or more 5-methylcytosines. In certain embodiments, each cytosine of an oligonucleotide or modified oligonucleotide comprises a 5-methylcytosine.

Certain Modifications

In certain embodiments, modified oligonucleotides described herein may comprise one or more modifications to a nucleobase, sugar, and/or internucleoside linkage, and as such is a modified oligonucleotide. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, a modified oligonucleotide comprises one or more modified nucleosides. In certain such embodiments, a modified nucleoside is a stabilizing nucleoside. An example of a stabilizing nucleoside is a sugar-modified nucleoside.

In certain embodiments, a modified nucleoside is a sugar-modified nucleoside. In certain such embodiments, the sugar-modified nucleosides can further comprise a natural or modified heterocyclic base moiety and/or a natural or modified internucleoside linkage and may include further modifications independent from the sugar modification. In certain embodiments, a sugar modified nucleoside is a 2′-modified nucleoside, wherein the sugar ring is modified at the 2′ carbon from natural ribose or 2′-deoxy-ribose.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

Nucleosides comprising such bicyclic sugar moieties are referred to as bicyclic nucleosides or BNAs. In certain embodiments, bicyclic nucleosides include, but are not limited to, (A) α-L-Methyleneoxy (4′-CH₂—O-2′) BNA; (B) β-D-Methyleneoxy (4′-CH₂—O-2′) BNA; (C) Ethyleneoxy (4′-(CH₂)₂—O-2′) BNA; (D) Aminooxy (4′-CH₂—O—N(R)-2′) BNA; (E) Oxyamino (4′-CH₂—N(R)—O-2′) BNA; (F) Methyl(methyleneoxy) (4′-CH(CH₃)—O-2′) BNA (also referred to as constrained ethyl or cEt); (G) methylene-thio (4′-CH₂—S-2′) BNA; (H) methylene-amino (4′-CH2-N(R)-2′) BNA; (I) methyl carbocyclic (4′-CH₂—CH(CH₃)-2′) BNA; (J) c-MOE (4′-CH(CH₂—OMe)-O-2′) BNA and (K) propylene carbocyclic (4′-(CH₂)₃-2′) BNA as depicted below.

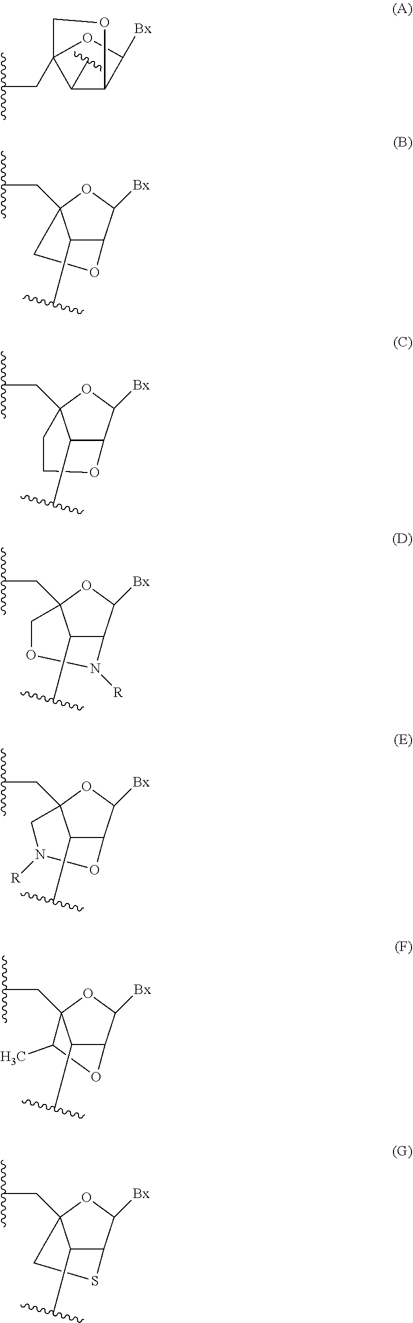

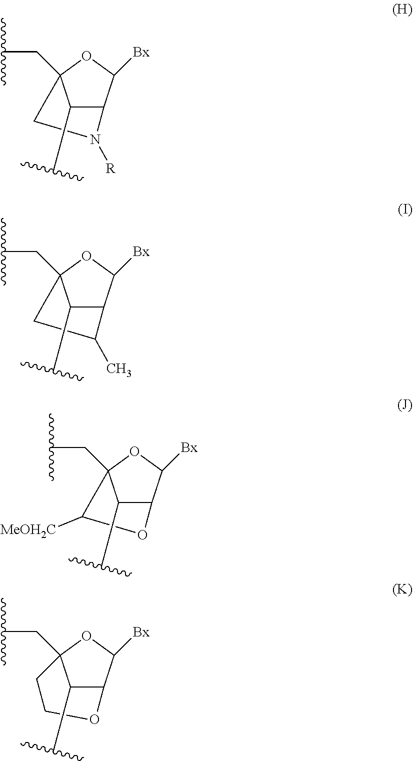

wherein Bx is a nucleobase moiety and R is, independently, H, a protecting group, or C₁-C₁₂ alkyl.

In certain embodiments, a 2′-modified nucleoside comprises a 2′-substituent group selected from F, OCF₃, O—CH₃, OCH₂CH₂OCH₃, 2′-O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-modified nucleoside comprises a 2′-substituent group selected from F, O—CH₃, and OCH₂CH₂OCH₃.

In certain embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4′-0 replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O—(CH₂)₂—OCH₃, and 2′-F.

In certain embodiments, a modified oligonucleotide comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In certain embodiments, each internucleoside linkage of a modified oligonucleotide is a phosphorothioate internucleoside linkage.

In certain embodiments, a modified oligonucleotide comprises one or more modified nucleobases. In certain embodiments, a modified nucleobase is selected from 5-hydroxymethyl cytosine, 7-deazaguanine and 7-deazaadenine. In certain embodiments, a modified nucleobase is selected from 7-deaza-adenine, 7-deazaguanosine, 2-aminopyridine and 2-pyridone. In certain embodiments, a modified nucleobase is selected from 5-substituted pyrimidines, 6-azapyrimidines and N-2, N-6 and O-6 substituted purines, including 2 aminopropyladenine, 5-propynyluracil and 5-propynylcytosine.

In certain embodiments, a modified nucleobase comprises a polycyclic heterocycle. In certain embodiments, a modified nucleobase comprises a tricyclic heterocycle. In certain embodiments, a modified nucleobase comprises a phenoxazine derivative. In certain embodiments, the phenoxazine can be further modified to form a nucleobase known in the art as a G-clamp.

In certain embodiments, a modified oligonucleotide is conjugated to one or more moieties which enhance the activity, cellular distribution or cellular uptake of the resulting antisense oligonucleotides. In certain such embodiments, the moiety is a cholesterol moiety. In certain embodiments, the moiety is a lipid moiety. Additional moieties for conjugation include carbohydrates, phospholipids, biotin, phenazine, folate, phenanthridine, anthraquinone, acridine, fluoresceins, rhodamines, coumarins, and dyes. In certain embodiments, the carbohydrate moiety is N-acetyl-D-galactosamine (GalNac). In certain embodiments, a conjugate group is attached directly to an oligonucleotide. In certain embodiments, a conjugate group is attached to a modified oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises a modified oligonucleotide having one or more stabilizing groups that are attached to one or both termini of a modified oligonucleotide to enhance properties such as, for example, nuclease stability. Included in stabilizing groups are cap structures. These terminal modifications protect a modified oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Certain Small Molecules

In certain embodiments, the inhibitor of the microRNA is a small molecule. Various small molecule miRNA inhibitors and methods for identifying such inhibitors have been reported. Small molecule miRNA inhibitors are discussed generally, e.g., in Jeker and Marone, Curr. Opin. Pharmacol. 2015; 23: 25-31. Streptomycin has been reported to inhibit miR-21 function. See Bose et al., Angew. Chem. Int. Ed. Engl. 2012; 51: 1019-1023. Certain small molecules comprising azobenzene functionality were reported as having mir-21 inhibitory activity. See Gumireddy et al., Angew. Chem. Int. Ed. Engl. 2008; 47(39): 7482-7484; see also Huang et al., US2014/0255386. Small molecule inhibitors and activators of miR-122 are reported in Young et al., J. Am. Chem. Soc., 2010; 132: 7976-7981 and additional miR-122 inhibitors are reported in Connelly et al., J. Biomol. Screen. 2012; 17: 822-828. Connelly et al. also report a high-throughput luciferase reporter screen for identifying small molecule miRNA inhibitors. Additional small molecules are reported in, e.g., Deiters, US2013/0005759 (miR-122); Chen, US2014/0179799 (miR-34a); Bose et al., ACS Chem. Biol. 2013; 8: 930-938 (miR-27a).

Additionally, methods have been reported for design of small molecules to target RNAs, such as precursor miRNAs, based on the target RNA sequence, wherein such molecules reportedly inhibit miRNA function. See Velagapudi et al., Nat. Chem. Biol. 2014; 10: 291-297; Disney et al., Org. Biomol. Chem. 2014; 12: 1029-1039 (see also Disney et al., WO2015/021415). A screening assay for small molecule regulators of miR-133 is reported in Rudnicki, US2014/0221466.

### EXAMPLES

The following examples are presented in order to more fully illustrate some embodiments of the invention. They should in no way be construed, however, as limiting the broad scope of the invention. Those of ordinary skill in the art will readily adopt the underlying principles of this discovery to design various compounds without departing from the spirit of the current invention.

### Example 1: RT-Interference Method Poorly Reflects Anti-miR Binding

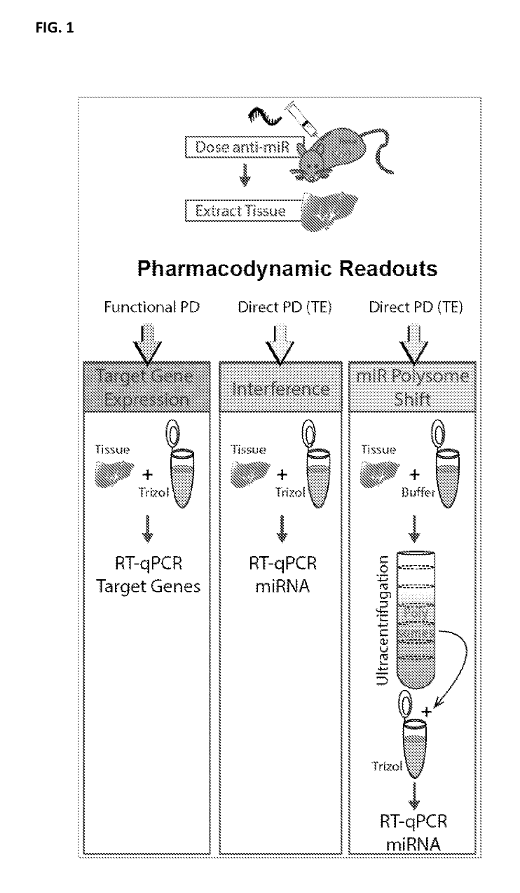

Measurement of microRNA inhibition by RT-interference has been considered convenient and simple (FIG. 1). To test the validity of the method for measuring anti-miR binding stoichiometry, synthetic microRNAs were annealed with complementary high-affinity anti-miRs. The fraction of microRNA detectable by RT-qPCR was measured to determine whether it reflected the expected annealed ratio.

microRNA:Anti-miR Duplex Formation

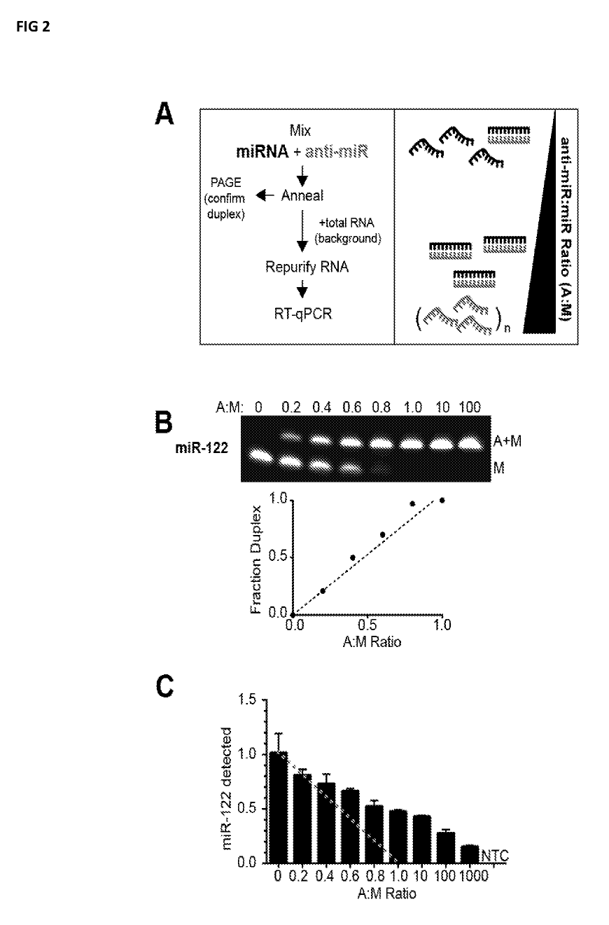

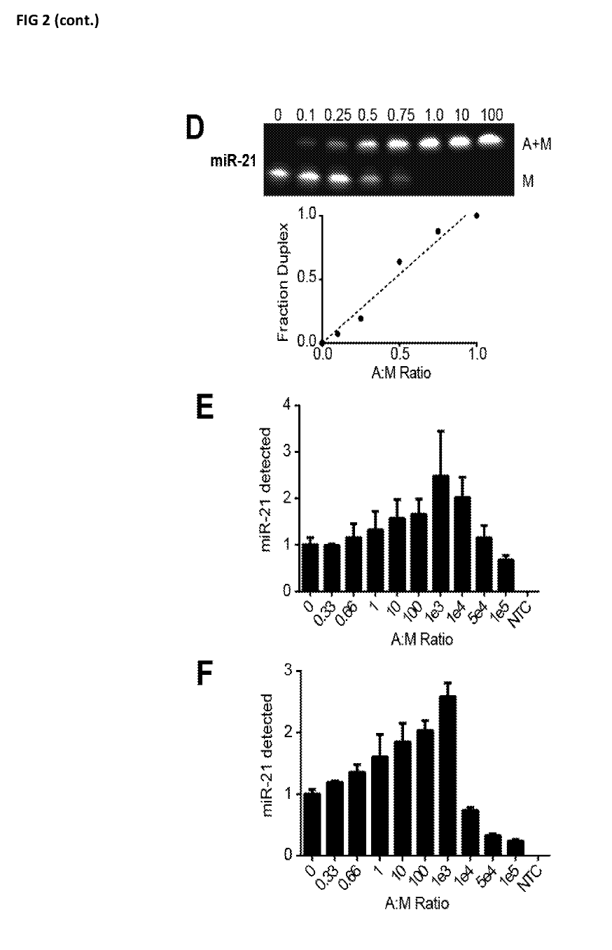

Synthetic microRNAs were annealed with complementary high-affinity anti-miRs in increasing ratios from sub-stoichiometric to up to 100,000-fold excess of anti-miR (FIG. 2A). Annealing efficiencies were confirmed using non-denaturing PAGE (FIGS. 2B and 2D). This process was performed for two microRNA plus anti-miR combinations: one using miR-122 the other with miR-21. Annealed samples were then added back to purified total RNA depleted of endogenous miR-122 and miR-21. Samples then underwent a second round of trizol/cartridge RNA purification to account for any loss of microRNA:anti-miR duplex into the acidic organic phase (Davis et al., Nucleic Acids Res., 2009; 37: 70-77).

RT-Interference

RT-interference was assessed using Taqman microRNA assays (Life Technology) with input of 30-100 ng total RNA extracted with RNeasy® 96-well spin plates from intact liver or exakidney tissue or from S16 lysates prepared as described above. For annealing experiments, synthetic guide strand (IDT) miR-122 or miR-21 (sequences from www.mirbase.org; see Griffiths-Jones, S., Nucleic Acids Res, 2004; 32: D109-111) were annealed with anti-miR in PBS using a thermal cycler with a programmed ramp down from 85° C. to 10° C. at a rate of 0.1° C./s. Annealed microRNA solutions were then diluted to 200 or 20 pM in a 200 μl volume containing 2 μg pre-purified total RNA isolated from kidneys of miR-21 null mice that was confirmed to contain neither miR-21 nor miR-122, the latter of which is not endogenously expressed in kidney (FIG. 2C,E,F). Samples then underwent a second round of RNA purification before RT-qPCR measurements. A parallel set of reactions was prepared using 3′-Cy3 conjugated guide strands that were then run on pre-cast 20% Novex® TBE polyacrylamide gels (Life Technologies). Annealing ratios were estimated based on relative top and bottom band intensities quantified using ImageJ software (NIH).

Quantification of Anti-miR

All work was carried out using high-affinity anti-miRs containing constrained ethyl (cET)-chemistries and phosphorothioate backbones. Anti-miR concentrations in sucrose gradient fractions were determined by hybridization with complementary fluorescent probes detected by high performance liquid chromatography (HPLC-FL) using an Agilent model G13321C fluorescence detector coupled to an Agilent 1260 series HPLC pump. Analysis of HPLC-FL signals was performed using MassHunter Version 7.0 (Agilent Technologies). HPLC-FL peaks were identified by comparing retention times to that of standards prepared by spiking known concentrations of anti-miR into sucrose solutions of matching density. Anti-miR concentrations in plasma samples were determined using a hybridization-based enzyme-linked immunosorbent assay (ELISA) (see Yu, R. Z., et al., Analytical Biochemistry, 2002; 304: 19-25). Briefly, a DNA probe containing biotin at one end and digoxigenin at the other was hybridized with analyte in plasma matrix and subsequently immobilized in a streptavidin-coated plate. Unhybridized probe was cleaved using a nuclease and then removed via a buffer wash. The digoxigenin-labeled probe was detected using anti-digoxigenin antibody conjugated to alkaline phosphatase, which catalyzed the formation of fluorescent AttoPhos® (Promega). Fluorescence intensity was determined using a fluorescence plate reader.

Results

Whether the fraction of microRNA detectable by RT-qPCR reflected the expected annealed ratio was evaluated. For miR-122, RT-qPCR measurements underestimated the ratio of microRNA bound to anti-miR (FIG. 2B-C). Approximately 50% of miR-122 was still detectable at a 1:1 ratio (FIG. 2C), where miR-122 was fully duplexed with an anti-miR-122 (FIG. 2B). Only at an excess of 1,000-fold did the microRNA signal begin to approach the level of background. These results suggest that RT primer can effectively compete off anti-miR for binding to microRNA.

In the results for miR-21 (FIG. 2D-F), excess anti-miR (A:M>10,000) caused negative interference (FIG. 2F), while lower A:M ratios (A:M≤10,000) caused a gain in signal (FIG. 2E-F). This positive interference effect amplified with additional anti-miR-21 up to A:M=10,000, which appeared to be the tipping point where the amplified signal either decreased back towards baseline or switched to a negative effect depending on the level of miR-21 included in the sample (FIG. 2E-F). Without wishing to be bound by theory, it is possible that an anti-miR-21, and perhaps other anti-miRs, can act as a template for PCR amplification.

Regardless of the mechanism of interference, these results demonstrate that, despite its convenience, the RT-interference approach appears to be a poor measure of microRNA inhibition by anti-miR that can produce inaccurate results.

### Example 2: Polysome Shift Assay: Validation with miR-122 in Mouse Liver

microRNAs associate with their targets in translationally active polyribosome complexes (polysomes). This association can be sensitive to translational inhibitors such as puromycin and is dependent on RNA-RNA interactions. Disruption of microRNA association with polysomes as a readout for anti-miR activity was tested using a microRNA Polysome Shift Assay (miPSA). Also tested were the effects of anti-miR-122 as a function of time, by comparing miPSA and target gene derepression.

microRNA Polysome Shift Assay

Frozen tissues, weighing 100-200 mg, were placed in Lysing Matrix D Fast-Prep Tubes (MP Biomedicals) containing 500 μl ice cold detergent-free buffer (10 mM Tris pH 7.4, 100 mM NaCl, 2.5 mM MgCl₂) supplemented with 100 μg/ml cycloheximide (EMD) and EDTA-free HALT® protease inhibitor cocktail (ThermoFisher). Samples were homogenized in a tissue homogenizer, shaking at 2,000 oscillations/min for 60-120 seconds. Resulting homogenates were cleared by centrifugation at 1000×g for 10 min at 4° C. Supernatants were then centrifuged twice at 16,000×g for 10 min at 4° C. Resulting S16 lysates were layered on top of 5-60% sucrose gradients and spun in an XL-90 ultracentrifuge at 40,000 rpm for 1.5 hours using a SW41 rotor (Beckman Coulter). Gradients were fractionated into 15 or 8 equal volumes by either a piston gradient fractionater (BioComp Instruments) or STAR-series liquid handling robot (Hamilton). Polysome-containing fractions were confirmed by in-line UV (254 nm) measurements or Quanti-iT® RiboGreen RNA reagent (Life Technologies). Polysome-containing fractions were then analyzed using microRNA TaqMan assays (Life Technologies) to quantify relative levels of the miR-of-interest (MOI; i.e. the microRNA being inhibited) and a reference (REF) microRNA. Relative displacement of the MOI normalized to the REF was then calculated between treated and control samples using the ΔΔCT method. Displacement values are reported in the log₂ scale such that positive values reflect loss of microRNA from polysome fractions (i.e. displacement=ΔΔCT).

Measurement of Target Gene Expression

Tissue samples, weighing ˜30 mg, were homogenized in Qiazol and total RNA was extracted using RNeasy® 96-well spin-plates as instructed by the manufacturer (Qiagen). RNA integrity was confirmed on an Agilent 2100 Bioanalyzer. Random cDNA was synthesized using a High Capacity cDNA Reverse Transcription kit with 50-200 ng RNA input (Life Technologies). After reverse transcription was complete, cDNA was diluted 1:3 with ddH2O and a 2.0 μl volume was used as input for each 10 μl qPCR reaction prepared with Universal TaqMan Master Mix II without UNG (Applied Biosystems) and TaqMan primer/probesets (IDT).

Results

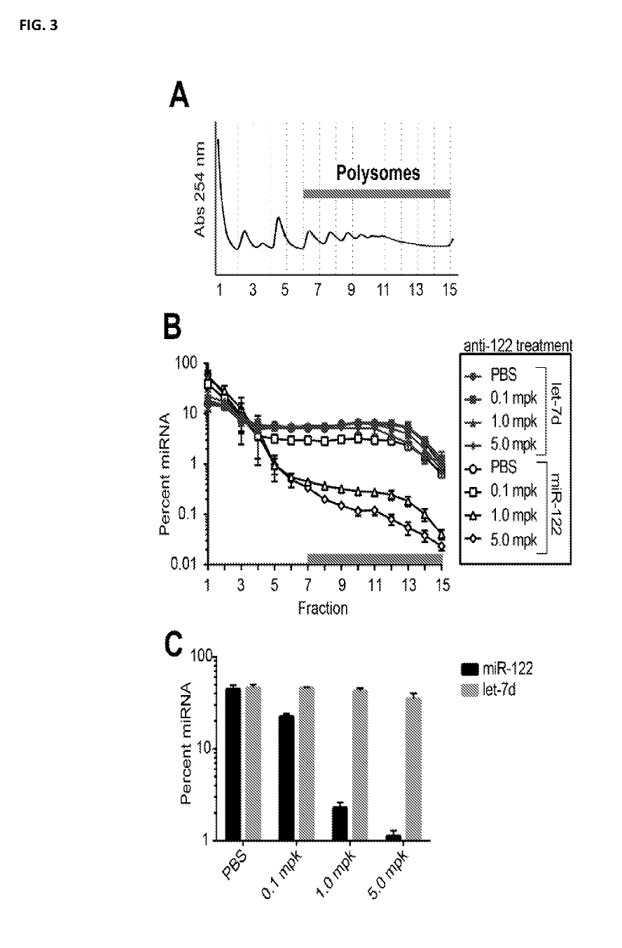

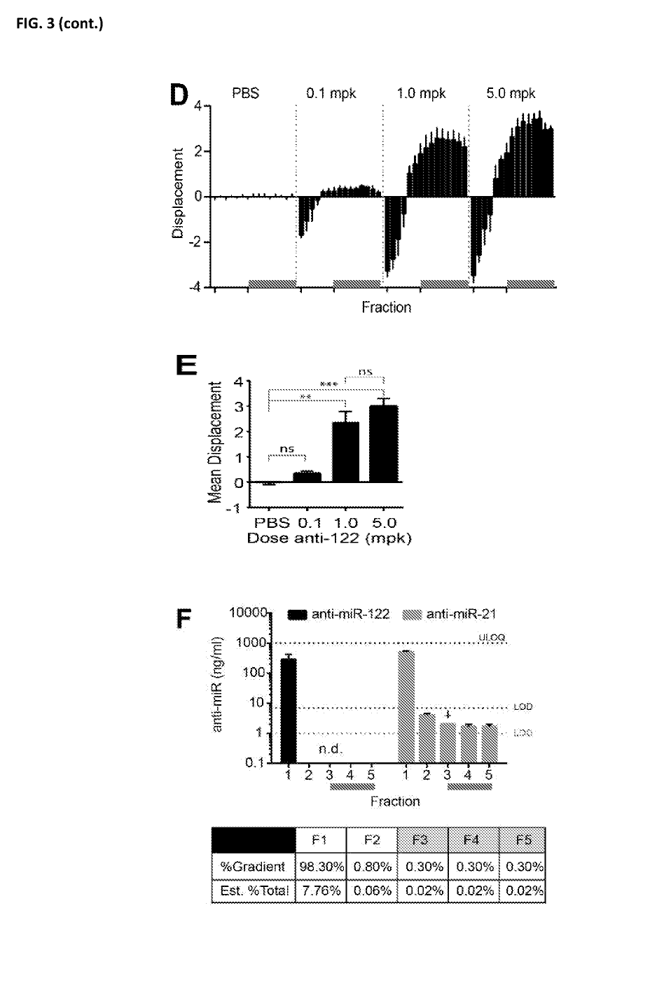

In untreated mouse liver, miR-122 was found to be well associated with polysomes (FIG. 3A-B). Upon treatment with an anti-miR-122 compound, the percentage of miR-122 in polysome fractions dropped in a dose-dependent manner from ˜44% to 1.2% after 24 hours (FIG. 3B-C). This effect was specific to miR-122 and did not occur with let-7d, a microRNA with similar basal polysome occupancy as miR-122 (FIG. 3C). To improve quantification and to standardize effect size, let-7d was used as a reference and displacement scores, analogous to ΔΔCT values obtained in the familiar 2^{-ΔΔCT} method for relative quantification of PCR, were calculated for each fraction (FIG. 3D).

Closer inspection of these normalized data revealed that for miR-122 lost in polysome fractions, a reciprocal gain in miR-122 was observed in light fractions. Recovery of miR-122 mass balance was incomplete, likely due to RT-interference effects from anti-miR-122 present in light fractions (FIG. 3F). Importantly, little-to-no anti-miR was detected in polysome fractions, thus enabling artifact-free quantification in these regions of the gradient (FIG. 3F). Summary statistics were then computed for each sample by taking the mean displacement across all polysome fractions (FIG. 3E). Alternatively, a subset of polysome fractions could be sampled to sufficiently estimate the mean, thus decreasing processing time. Represented this way, these data clearly showed a steep differential in shifts between 0.1 and 1.0 mg/kg body weight (mpk) dose levels, with less significant differences between the top doses of 1.0 and 5.0 mpk (FIG. 3E).

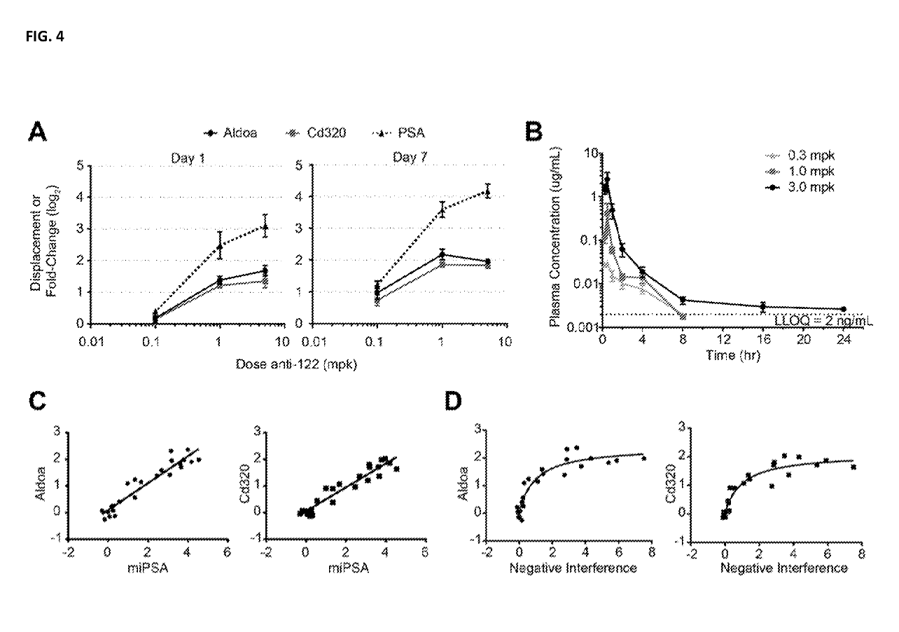

The miPSA and target gene derepression were compared at 1 and 7 days post treatment. At both time points, anti-miR induced dose-responsive derepression of miR-122 target genes Aldoa and Cd320 (FIG. 4A). Similar trends were observed in displacement of miR-122, but with a larger dynamic window than either of the genes-approximately 4-fold (linear) greater maximum response at the highest dose, where both measurements approached saturation (FIG. 4A). Overall displacement scores strongly correlated with target gene derepression (FIG. 4C). Additionally, both readouts similarly showed greater response at the day 7 time point compared to day 1 (FIG. 4A). Within 8 hours after injection >99.5% of the anti-miR-122 (relative to Cmax) was cleared from plasma and quickly taken up into hepatocytes (FIG. 4B). This suggests that while anti-miR levels in liver tissue are established shortly after treatment, additional time is required for anti-miR to reach the active site (see Koller, E., et al., Nucleic Acids Res, 2011; 39, 4795-4807; Wagenaar, T. R., et al., Nucleic Acids Res, 2015; 43, 1204-1215). That target genes and miPSA both show this delayed PD response strongly argues against miPSA displacement being induced by post-lysis leakage of otherwise inactive anti-miR oligonucleotides.

To measure RT-interference, microRNA levels were measured using TaqMan microRNA expression assays with input from RNA extracted directly from treated tissue (FIG. 1; Example 1). Unlike the linear trend observed with miPSA, RT-interference exhibited a hyperbolic relationship with target gene derepression (FIG. 4D): At low doses of anti-miR, RT-interference underestimated PD compared to target genes; while at high doses of anti-miR, RT-interference exaggerated PD after target gene response already saturated. These trends closely reflected those observed with annealed microRNAs in vitro (FIG. 2C).

Taken together, these results demonstrate that miPSA, but not RT-interference, can be used as a surrogate PD measure for target gene derepression, and can provide the advantage of a greater dynamic window than even the most robust target genes.

### Example 3: Measuring Anti-miR Pharmacology in Healthy Tissue

miR-21 is an attractive drug target, especially for kidney disease where its role has been validated through genetic knockout models (Gomez et al., JCI, 2015; 125: 141-156; Chau et al., Sci Trans Med., 2012; 4: 121ra1118). Under healthy conditions, however, miR-21 seemingly rests in an inactive state with minimal target gene repression (Chau et al., Sci Trans Med., 2012; 4: 121ra118; Androsavich et al., RNA, 2012; 18: 1510-1526), thus making it challenging to study anti-miR-21 inhibition by conventional means such as target derepression. To determine its applicability to the study of anti-miR activity in healthy tissue, miPSA was used to assess anti-miR-21 activity in normal mice.

Animal Care and Treatments

All animal experiments were conducted according to the Institutional AAALAC Guidelines. Male C57BL/6 mice (Jackson Laboratories) were housed four to five animals per cage with a 12 h light/dark cycle. Anti-miR oligonucleotides were dissolved in 1×PBS and administered to mice by subcutaneous injection at doses and frequencies described in the results section. At time of harvest, mice were humanely sacrificed by exposure to CO₂ or isoflurane (5% v/v), and euthanasia was confirmed by cervical dislocation. Dissected tissues were weighed and flash frozen in liquid nitrogen.

Estimation of miR-21 Expression in Liver

miR-21 expression in mouse liver was previously measured to be ˜400,000 copies per ng RNA (Meister et al., RNA, 2004; 10: 544-550; Krutzfeldt et al., Nature, 2005; 438: 685-689). Assuming 10 pg RNA per cell and 1×10⁸ hepatocytes per liver, there are then ˜4×10¹¹ copies of miR-21 in a mouse liver. Anti-miR-21 levels with a 45 mpk dose at day 7 were determined to be ˜30 ug/g or ˜3×10¹⁵ copies per 1 g mouse liver. This then gives anti-miR:microRNA (A:M)=3×10¹⁵/4×10¹¹=7500.

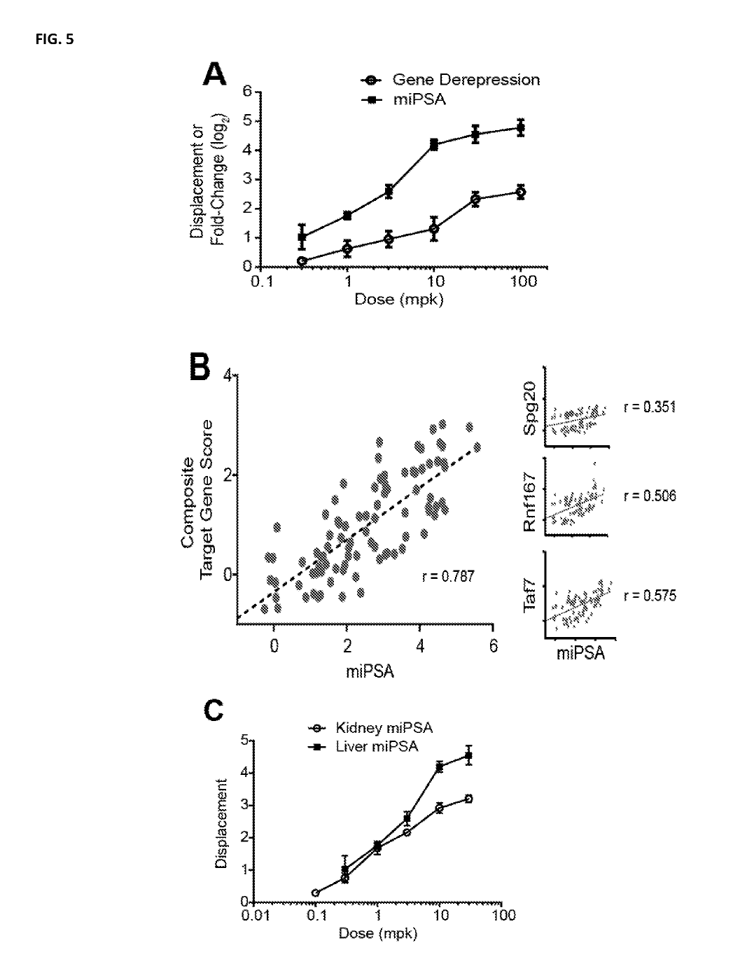

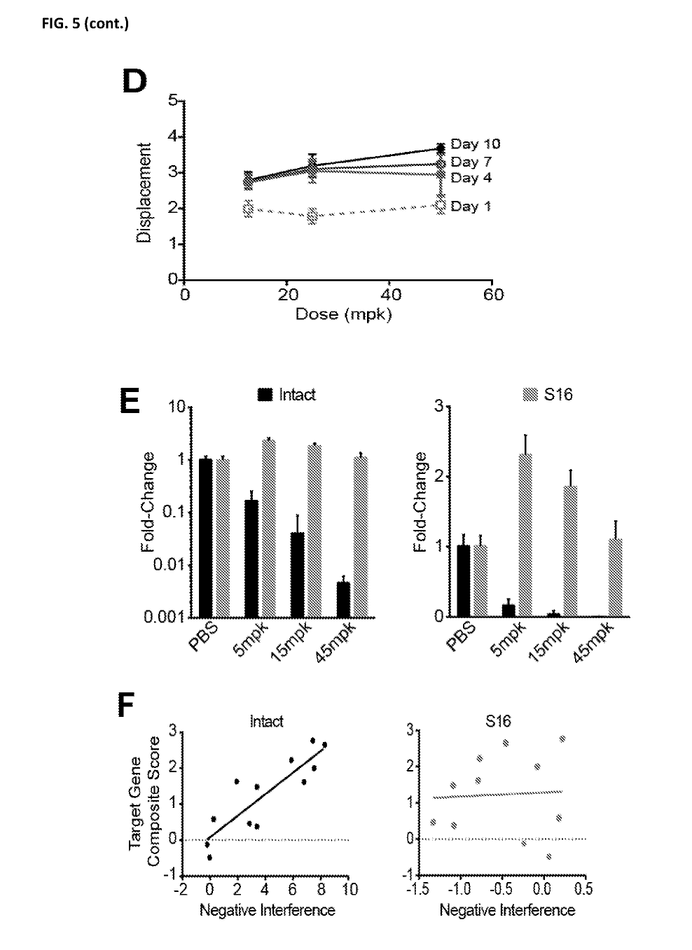

Treatment with anti-miR-21 compound resulted in a dose-responsive and specific shift in miR-21 from liver polysomes (FIG. 5A). Notably, the maximal shift observed at day 7 was comparable in magnitude to that of miR-122 (FIG. 4A), despite that miR-122 is more highly expressed and has greater polysome occupancy (Androsavich et al., RNA, 2012; 18: 1510-1526). This finding supports that microRNA polysome shift has a common upper limit related to maximal inhibition, thus making the readout both easy to interpret and comparable across different microRNAs, samples, and experiments.

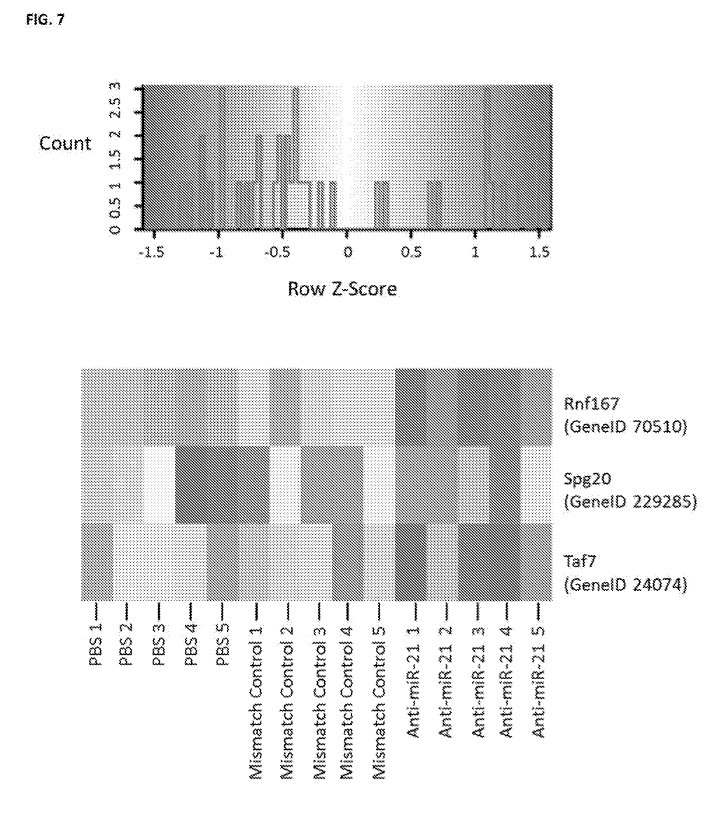

Despite lack of a global transcriptomic signature (Chau, B. N., et al., Science translational medicine, 2012; 4: 121ra118; Androsavich et al., RNA, 2012; 18: 1510-1526), in healthy liver tissue several target genes (all containing 3′UTR miR-21 seed-match sites) were identified that reproducibly showed weak fold-change in response to anti-miR-21 compounds but not a control mismatched anti-miR (FIG. 7). Summing log₂ fold-changes across these genes generated a composite score that was dose-responsive in liver (FIG. 5A). Again, miPSA provided an enhanced dynamic window while still correlating with target gene derepression (FIG. 5B). The correlation, however, was lower for miR-21 (r=0.787; FIG. 5B) than for miR-122 (r=0.9065; FIG. 4C), which may be attributed to additional noise in miR-21's less robust target genes. In agreement, higher correlation coefficients were observed using the miR-21 composite score compared to using any individual gene (FIG. 5B).

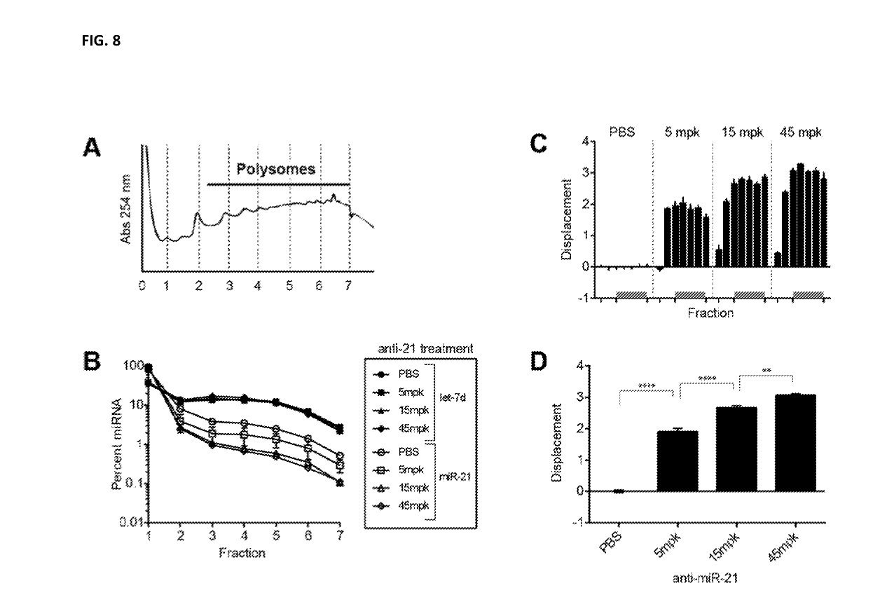

In kidney with low doses of anti-miR-21 (≤3 mpk), miPSA responses were similar to liver (FIG. 5C; FIG. 8). However, responses began to diverge at higher doses (>3 mpk), where kidney PD appeared to saturate at a lower maximal response (FIG. 5C). This suggests incomplete inhibition of miR-21 in total kidney tissue, consistent with known limitations in the ability of certain kidney segments to uptake oligo (Carome, M. A., et al., Nephron, 1997; 75: 82-87). Perhaps for the same reason, or due to tissue-dependent activities, measurable derepression of the miR-21 target gene signature was not reliably observed in unstressed kidney (data not shown). While this prevented a direct comparison, miR-21 shift in kidney displayed similarly delayed kinetics as to what had been previously observed for target genes (FIG. 5D; FIG. 4A). These data demonstrate that the miPSA can be a sensitive assay that enables measurement of microRNA inhibition even in healthy tissue with partial delivery of anti-miR, and/or where target gene response may be weak.

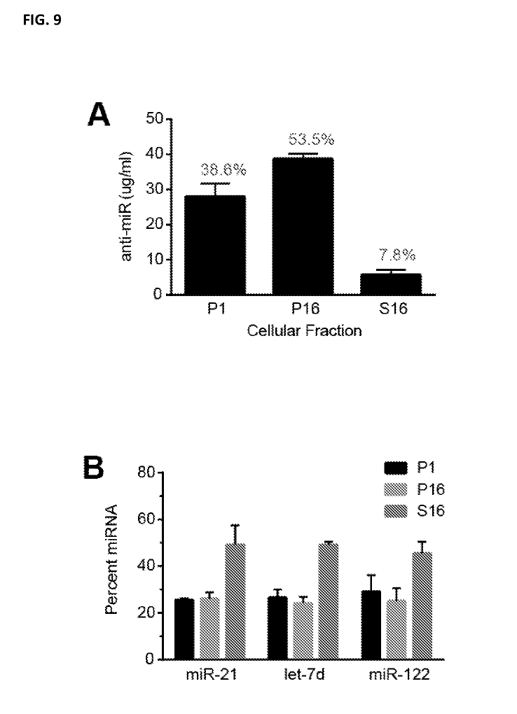

We again evaluated RT-interference for comparison. As described above, in vitro annealing experiments had shown a gain in miR-21 amplification in the presence of an anti-miR-21 (FIG. 2E-F). Using intact liver tissue treated in vivo, this same effect was not observed (FIG. 5E). Rather, RT-interference showed that increasing dosage of anti-miR-21 negatively interfered with miR-21 detection, and loss of miR-21 signal was found to linearly correlate with target gene derepression (FIG. 5F). Based on measurements of anti-miR tissue levels and miR-21 copies in liver (Androsavich et al., RNA, 2012; 18: 1510-1526; Bissels et al., RNA, 2009; 15: 2375-2384), we estimated ˜7,500-to-1 ratio of anti-miR-21 to miR-21 at the 45 mpk dose, approaching the upper limit tested in the in vitro annealing experiments where positive interference began to reverse (FIG. 2E-F). The same in vivo samples were again measured for RT-interference after first being fractionated into S16 lysates. In this manner the near majority (˜50%) of miR-21 and other microRNAs were preserved while anti-miR that excessively resides in non-productive compartments (Koller et al., Nucleic Acids Res., 2011; 39: 4795-4807; Wagenaar et al., Nucleic Acids Res., 2015; 43: 1204-1215) was depleted by >12-fold, reducing the estimated anti-miR:miR ratio to ˜1,000 at 45 mpk dose (FIG. 9). Under these conditions, RT-interference no longer correlated with target gene derepression (FIG. 5F). Moreover, at 5 and 15 mpk doses positive interference was again observed similar to in vitro annealed samples (FIG. 5E). With increasing dosage the enhancement in signal returned to neutral at 45 mpk, suggesting that the additional anti-miR present in lysates above that of the 5 mpk dose level began to tip interference back towards a negative effect.

In all, these data indicate that under some, but not all, conditions RT-interference can indeed correlate with functional readouts of microRNA inhibition; however, this outcome is largely driven by excess non-productive anti-miR and is not truly reflective of anti-miR:microRNA stoichiometry.

### Example 3: Assessing Intra- and Inter-microRNA Family Cross-Reactivity

miPSA's potential for assessing anti-miR specificity for target microRNAs with similar sequence was evaluated. It is assumed that anti-miRs will cross-react with microRNA family members sharing common seed motifs, since this region of the microRNA is the determining factor for target specificity (Lewis et al., Cell, 2003; 115: 787-798). This has been shown to be true for short seed-targeting anti-miRs using a luciferase reporter and pre-microRNAs co-transfected in succession (Obad et al., Nat Genet., 2011; 43: 371-378). Whether miPSA could be used to directly and simultaneously measure inhibition of individual native microRNA family members was tested.

Cell Culture

mIMCD-3 cells (ATCC, CRL-2123) were cultured in DMEM:F12 medium supplemented with 10% fetal bovine serum in 6-well culture plates. Anti-miRs were transfected with RNAiMax (Life Technologies) as per manufacturer's protocol. In preparation for miPSA, cells were incubated with cycloheximide (100 μg/ml) added to the growth media for 15-20 minutes at 37° C. After, cells were washed twice with ice-cold PBS with cycloheximide. 500 μl chilled Cell Lysis Buffer (20 mM HEPES, 125 mM KCl, 5 mM MgCl₂, 1× HALT® Protease inhibitor, 100 μg/ml cycloheximide, 100 U/ml RNAse Out (Life Technologies), 2 mM DTT, and 0.5% NP-40) was added to each well and the plate was set on top of ice for 10 minutes with occasional shaking to lyse the cells. Lysates were collected and cleared with a single bench top centrifugation step at 16,000×g for 10 min at 4° C. Supernatants were loaded on sucrose gradients and processed for miPSA as described below.

Results

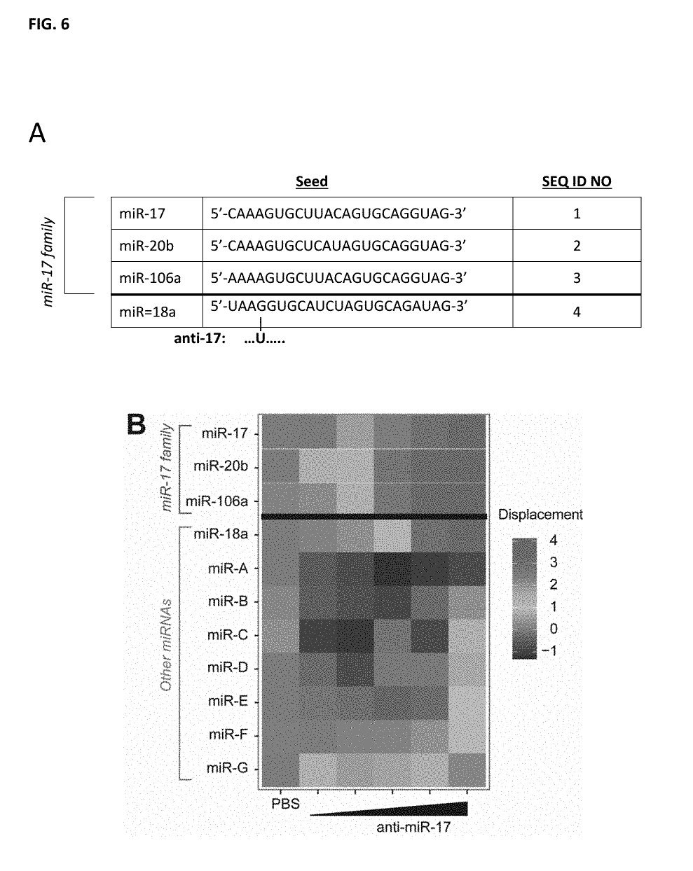

Cultured cells were transfected with increasing doses of an anti-miR-17, and subsequently analyzed with miPSA using primers to detect miR-17 and its seed-sharing family members miR-20b and miR-106a (FIG. 6A) along with additional non-related microRNAs. Consistent with inter-family cross-reactivity, all of the miR-17 family members were displaced from polysomes in a consistent dose responsive manner (FIG. 6B). Non-miR-17 family microRNAs, on the other hand, were unresponsive in comparison, with one exception: miR-18a showed strong cross-reactivity at higher doses (FIG. 6B).

Upon further inspection, a single nucleotide A/G difference between miR-17 and miR-18 seed sequences was found (FIG. 6A). It has previously been shown that a single point mismatch in the seed region is sufficient to disrupt anti-miR binding (Hogan, D. J., et al., PloS one, 2014; 9: e100951). However, this particular nucleotide switch would form a G:U wobble pair, a common RNA:RNA interaction (Varani, G. and McClain, W. H., EMBO Rep, 2000; 1: 18-23). Although the tolerability of G:U wobbles in microRNA targeting is unclear (Doench, J. G. and Sharp, P. A., Genes & Development, 2004; 18: 504-511; Lewis, B. P., et al., Cell, 2005; 120: 15-20; Miranda, K. C., et al., Cell, 2006; 126, 1203-1217), these non-Watson Crick base pairs are frequently found to be enriched in RNAs that co-immunoprecipitate with the central microRNA factor Ago (Chi, S. W., et al., Nature structural & molecular biology 2012; 19: 321-327; Clark, P. M., et al., Sci Rep, 2014; 4: 5947). This data suggests that permissiveness for at least one G:U wobble pair in the seed extends to chemically modified anti-miRs as well.

These data demonstrate that miPSA can also be a useful tool for quantifying anti-miR cross-reactivity.

### Example 4: Measuring Anti-miR Activity in an Accessible Tissue

In evaluating the activity of a microRNA inhibitor in a treated subject, it may be desirable to measure the extent to which the microRNA inhibitor engages with its target microRNA, particularly in the primary site of action of the microRNA. However, accessing tissue at the primary site of action may be difficult, or present a risk to the health of the subject. Additionally, the derepression of downstream target genes of the microRNA may not be a robust or reliable indicator of target engagement. The ability of the miPSA to address these challenges was evaluated.

The miPSA was used to test the ability of anti-miR-103/107 compounds to displace miR-103/107 from the polysome fraction of a treated sample obtained from and accessible tissue, and to determine the correlation of displacement value with efficacy in a model of impaired glucose tolerance and type 2 diabetes. Inhibition of miR-103/107 results in improved insulin sensitivity in a model of Type 2 diabetes.

Compounds were administered to high-fat fed obese mice (also called diet-induced obese mice or DIO mice), a model of impaired glucose tolerance and type 2 diabetes. Mice on a high fat (60% of Kcal from fat—Research Diet RD12492) were randomized into treatment groups based on similar baseline bodyweight, blood glucose and insulin. Three anti-miR-103/107 compounds, varying in nucleobase sequence, and placement and number of 2′-sugar modifications, were tested. Each compound was covalently linked to a conjugate moiety comprising three GalNAc residues, to enhance delivery of the compound to the liver.

In a first study, animals were treated with a single dose of PBS, anti-miR-1, or anti-miR-2. Anti-miR was administered via subcutaneous injection, at a dose of 60 mg/kg (mpk). Subcutaneous fat was collected 1, 3, and 7 days after treatment. The fat tissue was subjected to sucrose gradient fractionation, and the displacement value for each compound was determined, as described in Example 2. Derepression of seven different miR-103/107 downstream target genes was also measured in the fat tissue, as described in Example 2, and the individual changes in target gene expression were combined to give a composite score of downstream target gene derepression.

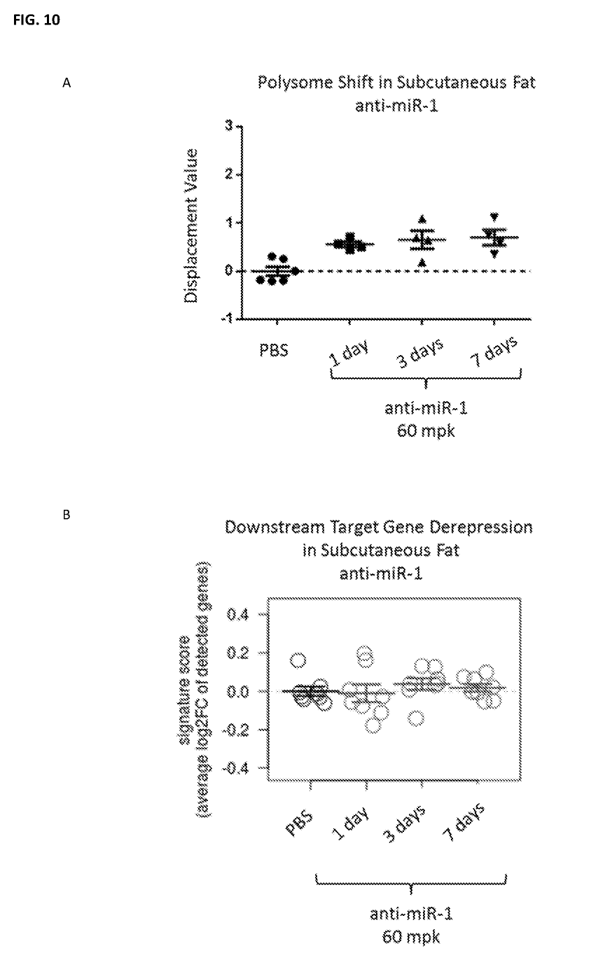

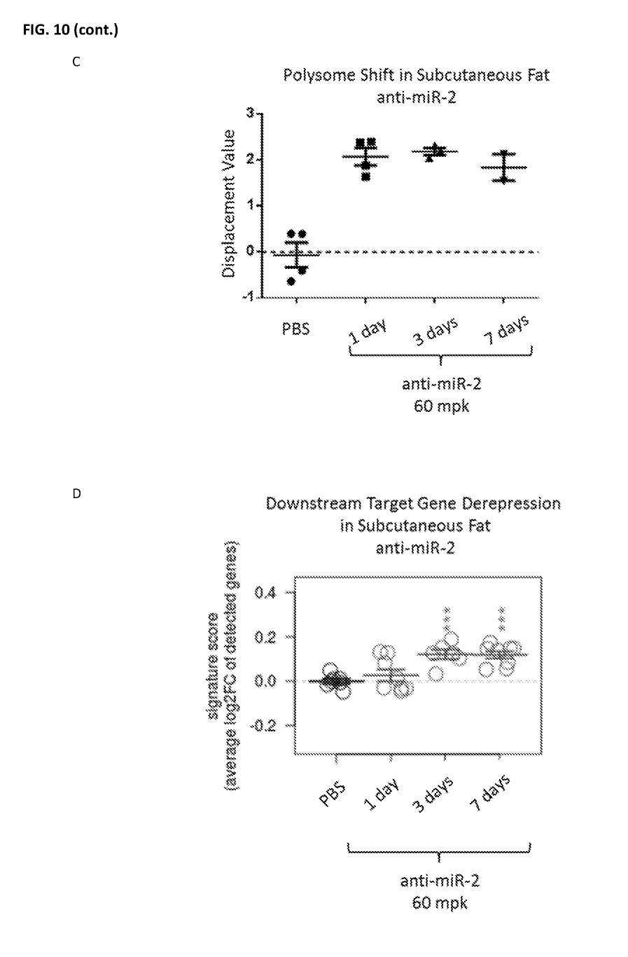

Displacement of miR-103/107 from the polysome fraction was detected in the fat tissue after a single administration of each anti-miR compound. As shown in FIG. 10A and FIG. 10B, anti-miR-1 exhibited displacement of miR-103/107 from the polysome fraction, but downstream target gene depression was not observed. As shown in FIG. 10C and FIG. 10D, anti-miR-2 also exhibited displacement of the target microRNA from the polysome fraction, however statistically significant downstream target gene derepression was observed. These data demonstrate that the miPSA can be used to detect microRNA inhibition in fat tissue, even when the fat tissue is not the primary site of delivery for the compound, and when downstream target gene derepression is not robust.

In a second study, the miPSA displacement values were compared to efficacy for anti-miR-1, at doses of 1.7, 5, 15, and 45 mpk, and anti-miR-3 at a dose of 15 mpk. Compound or PBS was administered to groups of 8 mice each, once weekly, for a total of 3 doses.

An oral glucose tolerance test (OGTT) was performed to determine how quickly glucose is cleared from the blood after glucose administration. After a 4 hour fast, blood was taken for measurements of fasting glucose and insulin. Next, 2 g/kg of dextrose is administered to each animal. Blood was then collected at 20, 40, 60 and 90 minutes, to measure glucose and insulin. The response to the glucose challenge was quantified by the area-under-the-curve method.

Subcutaneous fat tissue was collected 5 days following the final dose, and analyzed by the miPSA as described in Example 2.

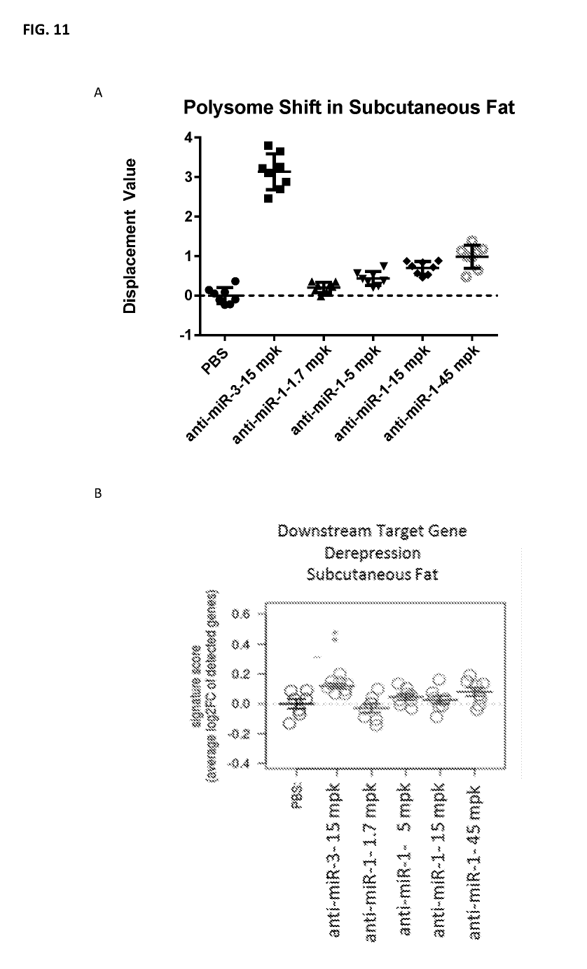

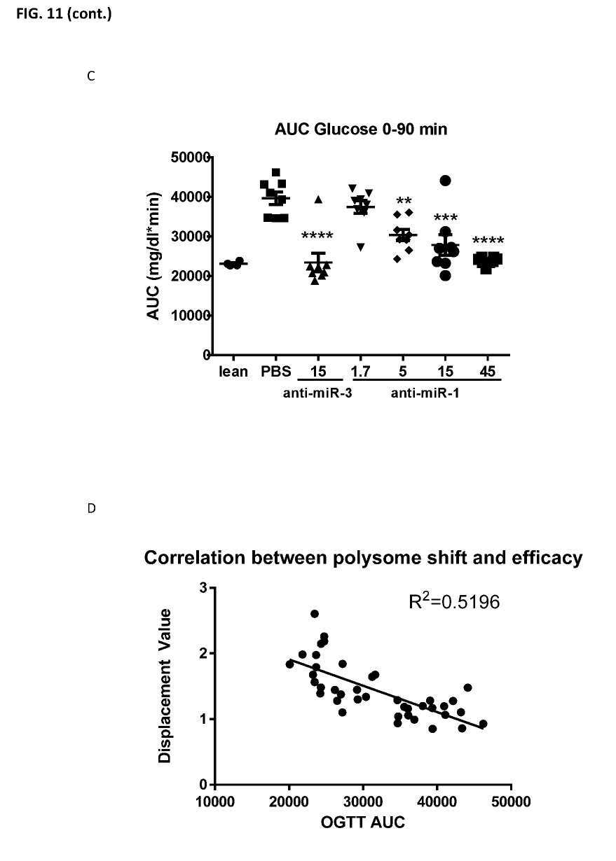

As shown in FIG. 11A, a dose-dependent loss of miR-103/107 from the polysome fraction was observed for anti-miR-1. Measurement of the derepression of seven different miR-103/107 downstream target genes showed a slight trend of target derepression for anti-miR-1, and a statistically significant derepression for anti-miR-3 (FIG. 11B). As shown in FIG. 11C, a dose-dependent improvement in efficacy was also observed. The calculated displacement values correlated with efficacy (FIG. 11D).

The seed region of miR-103/107 shares sequence similarity with members of the miR-15/16 family. As such, certain anti-miRs complementary to miR-103/107 may also hybridize to and inhibit the activity of a member of the miR-15/16 family. The miPSA was performed to determine whether it can identify anti-miRs which are cross-reactive with target microRNAs of similar sequence. miR-16 was chosen as a representative member of the miR-15/16 family.

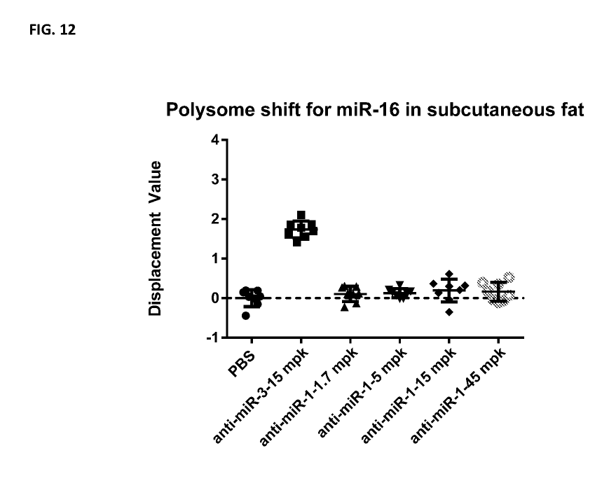

As determined by luciferase assay (data not shown), anti-miR-1 does not detectably inhibit miR-16, whereas anti-miR-3 does inhibit the activity of miR-16. As shown in FIG. 12, anti-miR-3 results in an appreciable polysome shift for miR-16, whereas anti-miR-1 does not. Thus, the miPSA may be used to identify anti-miRs that may be cross-reactive with non-target microRNAs.

These data demonstrate that the miPSA can be used to evaluate the efficacy of an anti-miR compound in an accessible tissue, such as adipose tissue. Further, these data demonstrate that the miPSA can identify anti-miRs that may be cross-reactive and inhibit the activity of non-target microRNAs.

### Example 5: Measuring Mimic Activity

The miPSA was used to test the activity of mimics. Mimics, like endogenous microRNAs, will associate with mRNAs in translationally active, high molecular weight polysome complexes, and thus be detected in the poly some fraction of a sample treated with a mimic.

Chemically modified, double-stranded mimics of miR-34a and let-7a were selected for testing in the miPSA. Both unconjugated compounds and compounds conjugated to cholesterol, a ligand that enhances delivery compound to the liver, were tested. The cholesterol moiety was conjugated to the active strand of the mimic (the strand with identity to the microRNA). Wild-type mice were treated with mimic compounds as follows:
30 mg/kg miR-34a mimic injected subcutaneously every 2 days for a total of 6 doses (n=4)
30 mg/kg miR-34a mimic injected subcutaneously every 2 days for a total of 3 doses (n=4)
30 mg/kg cholesterol-conjugated miR-34a mimic injected subcutaneously every 2 days for a total of 6 doses (n=4)
30 mg/kg cholesterol-conjugated miR-34a mimic injected subcutaneously every 2 days for a total of 3 doses
30 mg/kg let-7a mimic injected subcutaneously every 2 days for a total of 6 doses (n=4)
30 mg/kg let-7a mimic injected subcutaneously every 2 days for a total of 3 doses (n=4)
30 mg/kg cholesterol-conjugated let-7a mimic injected subcutaneously every 2 days for a total of 6 doses (n=4)
30 mg/kg cholesterol-conjugated let-7a mimic injected subcutaneously every 2 days for a total of 3 doses (n=4)
PBS injected subcutaneously (n=4)

Two days following the final dose, mice were sacrificed and liver and kidney tissues were harvested. The miPSA was performed as described herein. Amounts of microRNA detected were normalized to miR-16. An enrichment of microRNA in the translationally active polysome fraction is calculated as a negative displacement value.

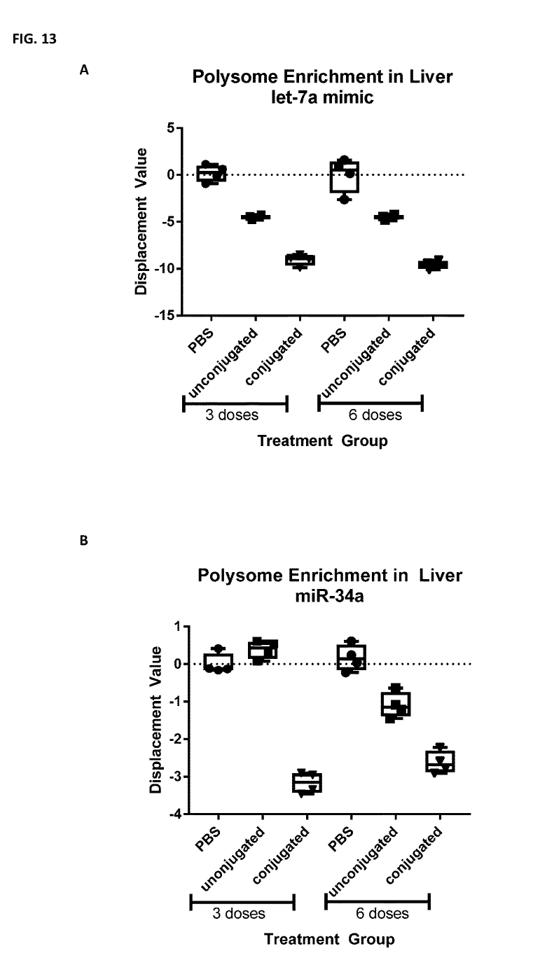

Treatment with a let-7a mimic, unconjugated or conjugated, resulted in enrichment of let-7a in the translationally active polysome fraction of sample isolated from liver (FIG. 13A). Similarly, treatment with a conjugated miR-34a mimic for 3 or 6 doses resulted in enrichment of miR-34a in the translationally active polysome fraction of sample isolated from liver (FIG. 13B). Enrichment was also observed following 6 doses of unconjugated miR-34a mimic. In samples isolated from kidney, enrichment was observed for the conjugated let-7a compound for both the 3 dose and 6 dose treatments (the cholesterol conjugate enhances delivery to liver, but does not eliminate some delivery to other tissues such as kidney). Enrichment following treatment with the unconjugated let-7a mimic was not observed in kidney. Enrichment of miR-34 in the polysome fraction of sample isolated from kidney following treatment with the miR-34a mimics was not observed.

These data demonstrated that the miPSA may be used to measure the activity of compounds that are mimics.

1. A method for determining the activity of an inhibitor of a target microRNA comprising:
a. determining a polysome occupancy in a treated sample;
b. determining a polysome occupancy in a control sample;
c. comparing the polysome occupancy in the treated sample to the polysome occupancy in the control sample to determine a displacement value for the inhibitor of the target microRNA, thereby determining the activity of the inhibitor of the target microRNA.

2. The method of claim 1, wherein the inhibitor of the target microRNA is a modified oligonucleotide.

3. (canceled)

4. The method of claim 1, wherein at least one downstream target of the microRNA is not measurably derepressed in the treated sample.

5. (canceled)

6. (canceled)

7. (canceled)

8. The method of claim 4, wherein the downstream target of the microRNA is a messenger RNA.

9. A method for determining the activity of a mimic of a target microRNA comprising:
a. determining a polysome occupancy in a treated sample;
b. determining a polysome occupancy in a control sample;
c. comparing the polysome occupancy in the treated sample to the polysome occupancy in the control sample to determine a displacement value for the mimic of the target microRNA, thereby determining the activity of the mimic of the target microRNA.

10. (canceled)

11. (canceled)

12. (canceled)

13. (canceled)

14. (canceled)

15. (canceled)

16. (canceled)

17. The method of claim 1, wherein the treated sample and control sample are each derived from a collection of cells.

18. The method of claim 1, wherein the treated sample and control sample are each derived from a tissue.

19. The method of claim 1, comprising preparing a lysate from the treated sample, and preparing a lysate from the control sample.

20. The method of claim 19, comprising separating the lysate from the treated sample into one or more polysomal compartments and one or more non-polysomal compartments, and separating the lysate from the control sample into one or more polysomal compartments and one or more non-polysomal compartments.

21. The method of claim 20, wherein the separating comprises:
a. layering the lysate on a sucrose gradient;
b. centrifuging the sucrose gradient; and
c. collecting one or more fractions of the sucrose gradient.

22. (canceled)

23. The method of claim 20, comprising identifying one or more polysomal compartments of the separated lysate.

24. The method of claim 1, wherein the method comprises determining at least one of the polysome occupancies by (i) separating at least one polysomal compartment of a sample from at least one non-polysomal compartment of that sample, (ii) quantifying the target microRNA in the separated polysomal compartment, and (iii) quantifying a reference RNA in the same separated polysomal compartment.

25. The method of claim 24, wherein quantifying the target microRNA and/or quantifying the reference RNA comprises quantitative PCR, spectrophotometry, electrophoresis, hybridization, precipitation, fluorometry, colorimetry, densitometry, scintillation counting, autoradiography, or a combination of two or more of the foregoing procedures.

26. The method of claim 24, wherein quantifying the target microRNA and/or quantifying the reference RNA comprises (a)(i) contacting the target microRNA and/or the reference RNA with a detectably labeled oligonucleotide to form a detection complex, and (ii) detecting the detection complex; or (b)(i) contacting the microRNA and/or the reference RNA with a primer, (ii) performing a nucleic acid synthesis reaction in which the primer is extended, and (iii) detecting nucleic acid produced by the nucleic acid synthesis reaction.

27. The method of claim 1, wherein the displacement value is determined by subtracting the logarithm of the control sample polysome occupancy from the logarithm of the treated sample polysome occupancy.

28. (canceled)

29. The method of claim 1, wherein the displacement value is determined as a logarithm of the quotient of control sample polysome occupancy divided by treated sample polysome occupancy.

30. (canceled)

31. The method of claim 1, wherein:
a) determining the polysome occupancy of the target microRNA in the treated sample comprises
i) measuring the amount of the target microRNA in the treated sample by quantitative PCR to generate a target microRNA Ct value for the treated sample;
ii) measuring the amount of a reference RNA in the treated sample by quantitative PCR to generate a reference RNA Ct value for the treated sample; and
iii) subtracting the reference RNA Ct value for the treated sample from the target microRNA Ct value for the treated sample, wherein the resulting value is the polysome occupancy for the treated sample;
b) determining the polysome occupancy of the target microRNA in the control sample comprises
i) measuring the amount of the target microRNA in the control sample by quantitative PCR to generate a target microRNA Ct value for the control sample;
ii) measuring the amount of a reference microRNA in the control sample by quantitative PCR to generate a reference RNA Ct value for the control sample; and
iii) subtracting the reference RNA Ct value for the control sample from the target microRNA Ct value for the control sample, wherein the resulting value is the polysome occupancy for the control sample.

32. The method of claim 31, wherein determining the displacement value for the inhibitor of the target microRNA comprises subtracting the polysome occupancy of the control sample from the polysome occupancy of the treated sample, wherein the resulting value is the displacement value for the target microRNA.

33. (canceled)

34. The method of claim 1, wherein the polysome occupancy is the amount of a target microRNA associated with a polysomal compartment normalized to the amount of a reference RNA associated with the same polysomal compartment.

35. The method of claim 34, wherein the reference RNA is selected from a reference mRNA and a reference microRNA.

36. (canceled)

37. (canceled)

38. (canceled)

39. (canceled)

40. (canceled)

41. (canceled)


SEQUENCE LISTING (4 sequences)

SEQ ID NO: 1 - RNA, 23 nt, Homo sapiens
caaagugcuuacagugcagguag

SEQ ID NO: 2 - RNA, 23 nt, Homo sapiens
caaagugcucauagugcagguag

SEQ ID NO: 3 - RNA, 23 nt, Homo sapiens
aaaagugcuuacagugcagguag

SEQ ID NO: 4 - RNA, 23 nt, Homo sapiens
uaaggugcaucuagugcagauag